SEQUENCE LISTING (34 sequences)

SEQ ID NO: 1 - protein, 10 aa, Artificial Sequence
  synthetic HSV-2 vaccine epitope
AEYDRVHIYY

SEQ ID NO: 2 - protein, 10 aa, Artificial Sequence
  synthetic HSV-2 vaccine epitope
AEIRDLARTF

SEQ ID NO: 3 - protein, 13 aa, Artificial Sequence
  synthetic HSV-2 vaccine epitope
AEIRDLARTFARE

SEQ ID NO: 4 - protein, 15 aa, Artificial Sequence
  synthetic HSV-2 vaccine epitope
WRQRLAHGRVRWVAE

SEQ ID NO: 5 - protein, 15 aa, Artificial Sequence
  synthetic HSV-2 vaccine epitope
HFTQYLIYDASPLKG

SEQ ID NO: 6 - protein, 14 aa, Artificial Sequence
  synthetic HSV-2 vaccine epitope
YLIYDASPLKGLSL

SEQ ID NO: 7 - protein, 13 aa, Artificial Sequence
  synthetic HSV-2 vaccine epitope
PMRARPRGEVRFL

SEQ ID NO: 8 - protein, 14 aa, Artificial Sequence
  synthetic HSV-2 vaccine epitope
ARLYPDAPPLRLCR

SEQ ID NO: 9 - protein, 11 aa, Artificial Sequence
  synthetic HSV-2 vaccine epitope
ARLYPDAPPLR

SEQ ID NO: 10 - protein, 10 aa, Artificial Sequence
  synthetic HSV-2 vaccine epitope
TIADFPLTTR

SEQ ID NO: 11 - protein, 9 aa, Artificial Sequence
  synthetic HSV-2 vaccine epitope
ASDSLNNEY

SEQ ID NO: 12 - protein, 15 aa, Artificial Sequence
  synthetic HSV-2 vaccine epitope
RLGPADRRFVALSGS

SEQ ID NO: 13 - protein, 15 aa, Artificial Sequence
  synthetic HSV-2 vaccine epitope
ITQFILEHRARASCK

SEQ ID NO: 14 - protein, 15 aa, Artificial Sequence
  synthetic HSV-2 vaccine epitope
TYGEMVLNGANLVTA

SEQ ID NO: 15 - protein, 13 aa, Artificial Sequence
  synthetic HSV-2 vaccine epitope
PMRARPRGEVRFL

SEQ ID NO: 16 - protein, 13 aa, Artificial Sequence
  synthetic HSV-2 vaccine epitope
RPRGEVRFLHYDE

SEQ ID NO: 17 - protein, 15 aa, Artificial Sequence
  synthetic HSV-2 vaccine epitope
DSGLLDYTEVQRRNQ

SEQ ID NO: 18 - protein, 15 aa, Artificial Sequence
  synthetic HSV-2 vaccine epitope
LDYTEVQRRNQLHDL

SEQ ID NO: 19 - protein, 15 aa, Artificial Sequence
  synthetic HSV-2 vaccine epitope
GPLGFLLKQFHAVIF

SEQ ID NO: 20 - protein, 15 aa, Artificial Sequence
  synthetic HSV-2 vaccine epitope
FLLKQFHAVIFPNGK

SEQ ID NO: 21 - protein, 15 aa, Artificial Sequence
  synthetic HSV-2 vaccine epitope
IAFIKRFSLDYGAIN

SEQ ID NO: 22 - protein, 15 aa, Artificial Sequence
  synthetic HSV-2 vaccine epitope
KRFSLDYGAINFINL

SEQ ID NO: 23 - protein, 68 aa, Artificial Sequence
  exemplary C4b multimerization domain
SGRAHAGWETPEGCEQVLTGKRLMQCLPNPEDVKMALEVYKLSLEIEQLELQRDSARQSTLDKELVPR

SEQ ID NO: 24 - protein, 55 aa, Artificial Sequence
  exemplary C4b multimerization domain
KKQGDADVCGEVAYIQSVVSDCHVPTAELRTLLEIRKLFLEIQKLKVELQGLSKE

SEQ ID NO: 25 - protein, 57 aa, Artificial Sequence
  exemplary C4b multimerization domain
ETPEGCEQVLTGKRLMQCLPNPEDVKMALEVYKLSLEIEQLELQRDSARQSTLDKEL

SEQ ID NO: 26 - protein, 58 aa, Artificial Sequence
  exemplary C4b multimerization domain
WETPEGCEQVLTGKRLMQCLPNPEDVKMALEVYKLSLEIEQLELQRDSARQSTLDKEL

SEQ ID NO: 27 - protein, 52 aa, Artificial Sequence
  exemplary C4b multimerization domain
CEQVLTGKRLMQCLPNPEDVKMALEVYKLSLEIEQLELQRDSARQSTLDKEL

SEQ ID NO: 28 - protein, 6 aa, Artificial Sequence
  6xhistidine tag
HHHHHH

SEQ ID NO: 29 - protein, 15 aa, Artificial Sequence
  Avitag amino acid sequence
GLNDIFEAQKIEWHE

SEQ ID NO: 30 - protein, 10 aa, Artificial Sequence
  synthetic HSV-2 vaccine epitope
MAEYDRVHIY

SEQ ID NO: 31 - protein, 9 aa, Artificial Sequence
  synthetic HSV-2 vaccine epitope
AEYDRVHIY

SEQ ID NO: 32 - protein, 15 aa, Artificial Sequence
  synthetic HSV-2 vaccine epitope
ERTIADFPLTTRSAD

SEQ ID NO: 33 - protein, 10 aa, Artificial Sequence
  synthetic HSV-2 vaccine epitope
RLYPDAPPLR

SEQ ID NO: 34 - protein, 9 aa, Artificial Sequence
  synthetic HSV-2 vaccine epitope
RPRGEVRFL

(19) United States
(12) Patent Application Publication
(10) Pub. No.: US 2020/0164060 A1
(43) Pub. Date: May 28, 2020

(54) HERPES SIMPLEX VIRUS VACCINE EPITOPES SPECIFICALLY RECOGNIZED BY TISSUE RESIDENT MEMORY T CELLS

(71) Applicants: Fred Hutchinson Cancer Research Center, Seattle, WA (US); University of Washington, Seattle, WA (US)

(72) Inventors: David Koelle, Seattle, WA (US); Anna Wald, Seattle, WA (US); Christine Johnston, Seattle, WA (US); Christine Posavad, Seattle, WA (US); Larry Corey, Mercer Island, WA (US)

(73) Assignees: Fred Hutchinson Cancer Research Center, Seattle, WA (US); University of Washington, Seattle, WA (US)

(21) Appl. No.: 16/632,853

(22) PCT Filed: Jul. 20, 2018
(86) PCT No.: PCT/US18/43137

§ 371 Date: Jan. 21, 2020

Related U.S. Application Data
(60) Provisional application No. 62/535,775, filed on Jul. 21, 2017.

(51) Int. Cl. A61K 39/245; C12N 7/00; C07K 14/005; C07K 14/705; C07K 14/73

(52) U.S. Cl. CPC A61K 39/245; C12N 7/00; C07K 14/005; C07K 14/70517; A61K 2039/572; C12N 2710/16634; C12N 2710/16622; C07K 2319/00; C07K 14/70514

(57) ABSTRACT

Herpes Simplex Virus type 2 (HSV-2) epitopes bound by CD8 or CD4 tissue resident memory cells at a healed site of HSV-2 infection are disclosed. The HSV-2 epitopes can be used as immunogenic compositions to elicit protective immune responses against HSV-2.

### CROSS-REFERENCE TO RELATED APPLICATION

This application claims priority to U.S. Provisional Patent Application No. 62/535,775 filed Jul. 21, 2017, the entire contents of which is incorporated herein by reference.

### STATEMENT OF GOVERNMENT SUPPORT

This invention was made with government support under A1091701, A1030731, and A1094019 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 18-007-WO-PCT Sequence Listing_ST25.txt. The text file is 11 KB, was created on Jul. 20, 2018, and is being submitted electronically via EFS-Web.

### FIELD OF THE DISCLOSURE

Herpes Simplex Virus type 2 (HSV-2) epitopes bound by CD8 or CD4 tissue resident memory cells at a healed site of HSV-2 infection are disclosed. The HSV-2 epitopes can be used as immunogenic compositions to elicit protective or therapeutic immune responses against HSV-2.

### BACKGROUND OF THE DISCLOSURE

Herpes Simplex Virus type 1 (HSV-1) and Herpes Simplex Virus type 2 (HSV-2) are viruses capable of infecting humans. HSV type 1 and 2 are significant causes of human morbidity. HSV-2 is sexually transmitted and is the causative agent of most recurrent genital herpes lesions. Infection with HSV-2 is associated with increased pregnancy risks that include spontaneous abortion, premature birth, and congenital infection of the newborn with the virus. In addition, infection with HSV-2 is also associated with an increased risk of HIV infection when exposed to HIV.

Unfortunately HSV-2 infections are often asymptomatic and most infected individuals are unaware they are infected. This ignorance of HSV-2 status is a major contributing factor to transmission to uninfected partners. It is estimated that in the USA, for example, from 40 to 60 million people are HSV-2 infected, with an incidence of 1-2 million infections and 600,000-800,000 clinical cases per year.

When HSV-2 infection occurs, the virus causes latent infection in sensory neurons in ganglia that enervate areas of the skin and mucosa. Periodically, the virus reactivates from latency and causes an active infection of the skin or mucosa in the areas that are enervated by the neuron with re-activated virus. Currently available therapies can decrease this lytic viral replication in the skin or mucosa. However, currently available therapies do not remove latent virus from infected sensory neurons. As a result, if an antiviral therapy is not being taken at the time of viral re-activation in the neurons, it will not reduce or prevent replication of the virus in the skin or mucosa, and thus, it is not able to reduce new symptoms or block the chance of shedding of live HSV-2 into the environment (and thus transmission of HSV-2). Current FDA licensed therapy can be taken on a continual basis (suppressive therapy), which reduces symptomatic outbreaks and HSV-2 shedding, but as soon as it is stopped, the same underlying pattern of recurrent symptoms and lesions returns.

A major goal of most vaccine design strategies is to elicit production of neutralizing antibodies, which are a type of antibody that can inhibit the biological function of its target. Neutralizing antibodies against viruses such as HSV-2 can function by blocking a virus from entering a cell.

However, for the chronic control of intracellular pathogens such as viruses, the T cell arm of the immune response is equally or more important than the antibody arm of the immune response. Newer concepts in vaccine development and T cell immunology are focused on tissue resident memory T cells or “TRM” cells. Briefly, TRM cells are left behind at sites of healed or resolved infection and are specific for the microorganism (e.g., HSV-2) that has been cleared by the immune system. These TRM cells stand as sentinels or guards and are early responders to a recurrence or re-infection. There are several recent review articles on these cells (PMID 27987416, 27618245, 26688350, 26282885, 25526394).

CD8 T cells are an important part of the host defense against HSV-1 and HSV-2. HSV-specific CD8 T cells permanently localize to sites of recurrent skin or eye infection even after recurrent infections heal, and per animal models provide local protection against recurrence. HSV-specific CD8 T cells also localize to the trigeminal ganglia (TG) in HSV-1 -infected humans, as well as experimental animals. The TG is the site of long-term latency and persistent infection in humans. Vaccines that elicit CD8 T cells can protect animals from HSV challenge. CD4 T cells are also known to be an important component of host defense. CD4 T cells also permanently localize to sites of previous HSV-1 and HSV-2 infection in humans (Zhu et al. (2009) Nature Medicine 15(8): 886; van Velzen et al. (2013) PLoS Pathogens 9(8): e1003547). CD4 T cells provide nutritive cytokines that support CD8 T cells and can also recognize and kill virally infected skin cells in the setting of local interferon upregulation, and also secrete cytokines such as interferon gamma that have antiviral properties.

### SUMMARY OF THE DISCLOSURE

The current disclosure provides vaccine epitopes that can be used to elicit or increase HSV-2-specific tissue resident memory (TRM) T cells at sites of primary or recurrent HSV-2 infection. The TRM-specific vaccine epitopes can be used to treat HSV-2 infection, reduce the risk or severity of HSV-2 infection, and/or induce an immune response against HSV-2.

In particular embodiments, the elicited or increased T cells are activated CD8 TRM T cells. In particular embodiments, the elicited or increased T cells are activated CD4 TRM T cells. In particular embodiments, the elicited or increased T cells are activated CD8 T cells and activated CD4 T cells.

In particular embodiments, the vaccine epitopes can be used to elicit or increase a local protective memory T cell population within a tissue. In particular embodiments, the activated T cells are recruited to a desired anatomic location. In particular embodiments, the vaccine epitopes can localize vaccine-elicited, HSV-2-reactive CD8 T and/or HSV-2-reactive CD4 T cells to the reproductive tract. In particular embodiments, the vaccine epitopes can localize vaccine-elicited, HSV-2-reactive CD8 T and/or HSV-2-reactive CD4 T cells to the genital skin. In particular embodiments, T cells including T cell receptors (TCRs) that bind HSV-2 epitopes are used in adoptive T cell transfer into a subject to treat HSV-2 infection.

The elicited or increased CD8 and/or CD4 memory T cells can persist in a subject for an extended period of time, for example, for at least 2 weeks, at least 1 month, at least 2 months, at least 3 months, at least 4 months, at least 5 months, at least 6 months, at least 1 year, at least 2 years, at least 3 years, at least 4 years, or at least 5 years.

### BRIEF DESCRIPTION OF THE SEVERAL VIEWS OF THE DRAWINGS

Many of the drawings submitted herein are better understood in color, which is not available in patent application publications at the time of filing. Applicants consider the color versions of the drawings as part of the original submission and reserve the right to present color images of the drawings in later proceedings.

FIG. 1. Identification methods for HSV specific CD8s from peripheral blood mononuclear cells (PBMC). A schematic outline of the reactive T cell identification process detailed in Jing et al. (2012) J. Clin. Invest 122(2): 654-673 is shown. PBMCs were obtained and CD14+cells were enriched and turned into dendritic cells (DC) by culture with GM-CSF and IL-4. HSV-infected HeLa cell preparations treated to optimize cross presentation were added to these DC. Then CD8 T cells from a patient were added to the DC, except that instead of adding CD8 T cells from PBMCs as in Jing et al. (2012), cells from a cervical or skin biopsy from a patient with genital HSV-2 infection were added. To concentrate on TRM, the cervical or skin biopsy was obtained after resolution of a recurrent HSV-2 infection. After co-incubation of a single cell preparation of cells from the biopsy with the HSV-2-laden DC, activated cells were sorted using CD137 as the activation marker. The putative HSV-2-reactive T cells were expanded and then tested for functional activity including validation of HSV-2 recognition and determination of fine epitopes.

FIG. 2. Schematic depicting principles of a viral genome-wide CD8 screen. A plasmid containing the coding sequence of an HLA cDNA molecule under the control of a strong promoter was added to Cos-7 cells, which are monkey cells that express monkey beta 2 microglobulin. The HLA molecular variant was chosen to match the patient being studied. The Cos-7 cells were simultaneously co-transfected with a plasmid containing DNA encoding a single HSV-2 gene. As there are >75 HSV-2 genes, there are >75 plasmids encoding these HSV-2 genes. Each plasmid encoding a single HSV-2 gene was co-transfected with the HLA molecule in separate microwells. In the Cos7 cells, peptides from the HSV-2 protein assemble onto the HLA molecule. The T cells from the biopsy were added, shown on the right of FIG. 2. If the TCR on the T cell recognizes the HSV-2 peptide presented by the HLA molecule, the T cell is activated. T cell activation was measured by IFN-gamma (IFN-γ) secretion.

FIG. 3. Information related to female subjects. These subjects were the donors of the skin and cervix biopsies used to discover TRM epitopes.

FIGS. 4A, 4B. HSV-2-reactive CD4 and CD8 T-cells are abundant in cervix ex vivo. FIG. 4A shows the gating scheme to allow isolation of the biopsy-derived CD4 T cells or CD8 T cells separately and to remove from analysis other cells including the dendritic cell preparation. FIG. 4B shows results of expression of CD137, an activation marker expressed on the surface of the biopsy T cells after they are activated through their T cell receptor, when the biopsy cells are co-incubated with DC that are either treated with mock virus (top) or HSV-2 (bottom) in the form of infected HeLA cells prepared for cross-presentation as outlined in Jing et al. J Clin Invest 2012. The left column shows biopsy CD4 T cells and the right column shows biopsy CD8 T cells.

FIG. 5. T_{RM} direct net ex vivo reactivity to whole HSV-2. This table summarizes HSV-2-reactive data for biopsy-derived CD4 or CD8 T cells. The biopsies were obtained from cervix or skin from the indicated subjects (ID numbers in left-most column). Some subjects had more than one biopsy date (dates not shown).

FIG. 6. HSV-2-reactive CD4 and CD8 T-cells: very abundant in cervix ex vivo in this specimen. The two dot plots in the left-most column are introductory plots that show the gating of CD3+CD4+ T cells (also called CD4 T cells) in the upper panel, or CD8 T cells in the lower panel. These T cells were obtained from a cervix biopsy. The ‘mock’ column shows the expression of the activation marker CD137 on the surface of the gated CD4 or CD8 T cells in response to mock virus. The ‘HSV-2’ column shows the expression of CD137 on the cell surface of gated CD4 or CD8 T cells from the cervix biopsy after exposure to HSV-2 in the form of DC treated with infected HeLa cells. A much higher proportion of the T cells express CD137 when they were exposed to DC treated with HSV-2-infected HeLa cells. The flasks at right show schematically the process of expansion of the sorted CD137-high cells that were physically separated from the CD4 T cells and from the CD8 T cells using a cell sorter.

FIG. 7. QC check CD8 T_{RM} to whole HSV-2 186. CD137 high and CD137 negative CD8 T cells were isolated from a skin or cervix biopsy and expanded in culture using the procedures described below. The cell culture origins are indicated at right. After two weeks, the presence of HSV-2-infected T cells was checked by using whole HSV-2 ('HSV-2′ column), negative controls (‘Mock’ and ‘Medium’ columns) or a positive control (‘PHA’ column).

FIG. 8. QC check CD4 T_{RM} to whole HSV-2 186. CD137 high and CD137 negative CD4 T cells were isolated from a skin or cervix biopsy and expanded in culture using the procedures described below. Whole HSV-2 was added as killed virus, with self, autologous PBMC added as antigen presenting cells (APC). The APC were dump-gated for this data presentation. The cell culture origins are indicated at right. After two weeks, the presence of HSV-2-infected T cells was checked by using whole HSV-2 ('HSV-2′ column), negative controls (‘Mock’ and ‘Medium’ columns) or a positive control (‘PHA’ column).

FIG. 9. HSV-2 proteome-wide CD8 T_{RM} screen for HLA A, B. Methods: Jing et al. (2016) J. Immunol. 1502366. Representative data for skin biopsy derived cells (left 4 graphs) and cervix biopsy derived cells (right 4 graphs). Each row is one of the subject's 4 individual HLA A or B allelic variants. Humans are diploid and each human has two HLA A variants and two HLA B variants with a few exceptions. Therefore, each of the H LA A and B variants for each subject was screened, for a total of 4 screens per biopsy. The X axis of each of the 8 graphs lists each individual HSV-2 gene. Cos7 cells were co-transfected as outlined in FIG. 2 with both an HLA gene and an HSV-2 gene. After adding bulk cervix or skin CD137-high cells, supernatant fluid was collected and IFN-γ was measured using ELISA. The ELISA values are on the y axis of each of the 8 graphs. The text in each graph shows the name(s) of the reactive HSV-2 gene(s) or gene fragment(s).

FIG. 10. Pathways to CD8 epitopes 1: prediction. For this example, the CD137 high cells from a biopsy were activated in response to HLA B*4402 and HSV-2 gene UL6. The peptides shown on the X axis were made based on a predictive algorithm for binding to HLA B*4402. Each peptide was tested at a concentration of 1 μg/ml in the same T cell activation assay described in FIG. 9, using HLA B*4402-expressing antigen presenting cells.

FIG. 11. Pathways to CD8 epitopes 2: ORF-covering peptides pools→single peptide validation. For this example, biopsy-derived cells recognize Cos7 co-transfected with the combination of a subject HLA molecule and HSV-2 UL25 gene (not shown). The UL25 peptides (15 mers, overlapping by 11 AA) were arrayed into a rectangular matrix, and row and column pools were created and tested such that each peptide was present at 1 μg/ml. The antigen presenting cells were HLA-expressing cells, the responder cells were the bulk biopsy-derived cells, and the readout was IFN-γ secretion. As shown in the graph at top, only one row and one column pool were positive. The single peptide at the intersection of the row and column pools, indicated at lower right (‘UL25 189-203’, ERTIADFPLTTRSAD, SEQ ID NO: 32), was tested by intracellular cytokine secretion as shown in the bottom, right-hand graph. The peptide elicited an IFN-γ response from the biopsy cells while media did not (bottom, left-hand graph).

FIG. 12. EC₅₀ examples of polyclonal responders. In these assays, bulk biopsy T cells were used as responder cells and indicated HLA (HLA B*4402)-expressing cells were used as antigen presenting cells. The indicated peptide in each graph was added at concentrations in molar shown on each X axis. The concentration eliciting 50% IFN-γ release, as indicated on the Y axis, approximates the EC₅₀ or concentration of peptide required for 50% triggering of the T cells.

FIG. 13. HSV-2 proteome-wide CD4 TRM screen. Methods: Johnston et al. (2014) J. Virol. JVI-03285. In this example, bulk CD4 T cells from a biopsy from the CD137-high fraction were expanded and tested against every HSV-2 protein. The names of the HSV-2 proteins are on the X axis. The antigen presenting cells were self PBMC that were treated with gamma irradiation to reduce or prevent proliferation. After 3 days of co-incubation of the biopsy CD4 cells, the antigen presenting cells, and the indicated HSV-2 proteins, the proliferation of the biopsy cells was measured using a radiation-based test indicated on the Y axis. A high value indicates proliferation, which indicates activation of CD4 T cells. At right, two positive controls are PHA and also whole inactivated HSV-2 viral antigen. The arrow indicates that the biopsy CD4 T cells reacted to full-length HSV-2 UL19 protein.

FIG. 14. CD4 epitopes: peptide pools→singles. For this example, biopsy-derived cells recognize Cos7 co-transfected with the combination of a subject HLA molecule and HSV-2 UL19 gene. The biopsy cells reacted to whole full length UL19 as shown in FIG. 13. A rectangular matrix of peptides covering UL19 was created and row and column pools were made as indicated on the X axis of the top graph. Only one row pool and one column pool gave a positive proliferative response using the assay described in FIG. 13. The peptide at the intersection of the positive row and column pools, UL19 305-319 (SEQ ID NO: 14), was tested at 1 μg/ml using HLA-expressing cells as antigen presenting cells and biopsy-derived HSV-2-specific CD4 TRM as responder cells. The readout was IFN-γ secretion, as shown in the bottom graphs.

### DETAILED DESCRIPTION

Herpes simplex virus type 1 (HSV-1) and Herpes simplex virus type 2 (HSV-2) are two members of the HSV family of α-herpesviruses, which establish lifelong latent infection in sensory neurons and lead to chronic herpes disease. HSV-1 infection causes facial/ocular disease, while HSV-2 is the leading cause of genital herpes, although both viruses can be found at oral and genital sites.

Although many, if not most, HSV-2 infections are asymptomatic or unrecognized, symptomatic primary genital HSV infection is characterized by vesicular and ulcerative skin lesions, which can result in neurologic and urologic complications. During initial infection, a long term persistent or latent infection is established in ganglion neurons, which can reactivate and cause recurrent genital disease or asymptomatic viral shedding. Recurrent herpes infection is a chronic, intermittent disease characterized by both symptomatic and asymptomatic periods of viral replication in the epithelial cells at mucosal sites or other peripheral sites. (Koelle D M et al., J Immunol. 166:4049, 2001). Both symptomatic and asymptomatic herpes infection can lead to transmission to seronegative individuals. Indeed, most transmission occurs during periods of unrecognized shedding. One of the most serious complications of genital herpes occurs when the virus is transmitted from mother to neonate. Infection of the neonate causes significant morbidity and mortality, even with proper antiviral therapy (Sacks S L et al., Antiviral Res. 63S1:S27, 2004). Genital herpes infection also increases the risk of acquiring HIV infection and increases shedding of HIV in genital lesions (Hughes et al. (2012) Journal of Infectious Diseases 205(3): 358-365; Celum et al. (2010) New England journal of medicine 362(5): 427-439; Zhu et al. (2009); Posovad et al., Proc Natl Acad Sci USA. 94:10289, 1997).

HSV-2 infection induces both humoral and T-cell mediated immunity; however, the mechanisms that contribute to long term control of genital herpes are not understood. Studies from animal models of HSV infection and human studies indicate that high levels of neutralizing antibodies and innate immunity (natural killer (NK) cells, interferon, and macrophages) contribute to protection from HSV infection but the major determinants of HSV protection are both CD4 and CD8 T cells (Ahmad A et al., J Virol. 74(16): 7196-7203, 2000; Aurelian L et al., J Gen Virol. 68:2831, 1987; Milligan G N et al., J Immunol. 160(12):6093, 1998.; Koelle et al, 1998). Clearance of virus from recurrent lesions is more closely correlated to T cell immunity. Thus, when a recurrent lesion occurs, mononuclear cells, primarily CD4 T cells, infiltrate the lesion as early as two days after formation, followed by an influx of CD8 T cells at later times (Cunningham A L et al., J Clin Invest. 75:226, 1985). Although both HSV-specific CD4 and CD8 T cell responses are detected, clearance of HSV-2 from lesions correlates with a CD8+ cytotoxic T lymphocyte (CTL) response (Koelle et al., J Clin Invest. 101:1500, 1998).

Newer concepts in vaccine development and T cell immunology are focused on tissue resident memory or “TRM” cells. Briefly, TRM cells are left behind at sites of healed or resolved infection and are specific for the microorganism (e.g., HSV-2) that has been cleared by the immune system. These TRM cells stand as sentinels or guards and are early responders to a recurrence or re-infection. There are several recent review articles on these cells (PMID 27987416, 27618245, 26688350, 26282885, 25526394). In the HSV context, several animal studies have shown that HSV-specific CD8 TRM that are left behind at sites of resolved HSV infection can reduce or prevent re-infection (Shin & Iwasaki (2012) Nature, 491(7424): 463). Zhu et al. (2007) identified the first human HSV TRM and showed that the TRM are novel by their genetic CDR3 locus (Zhu et al. (2007) Journal of Experimental Medicine 204(3): 595-603).

More particularly, it was published in Nature in 2012 that a vaccination strategy in mice that produced CD8 T cells but not antibody or CD4 T cell responses, and localized the CD8 T cells to the reproductive tract, could protect mice from a vaginal HSV-2 challenge infection (PMID 23075848).

There is a therapeutic vaccine for genital HSV-2 infection that has shown 50% efficacy in phase II clinical trials (Genocea Biosciences Gen-003, see PMIDs 28329211, 27642130). This vaccine elicits CD8 T cell responses in addition to other types of immune responses. This is the first candidate therapeutic vaccine that is therapeutically active in clinical trials. It contains regions of genes US6 and RS1. There is also a therapeutic vaccine under development by Immune Design Corporation in Seattle that contains parts of genes US6, UL25, and UL19. Animal data related to the development of this vaccine is described in PMID 26571309.

As indicated previously, human HSV-2 genital infection has an intermittent recurrent pattern. After a genital herpes recurrence, the skin heals and appears normal. In a 2013 Nature paper (PMID 23657257), it was shown that HSV-2-specific CD8 TRM are left behind in normal-appearing, healed skin at the site of, and after, the resolution of an HSV-2 episode. CD4 TRM are also observed in healed skin after HSV-2 resolution (PMID 19643807; see also PMID 25170048).

The current disclosure provides HSV-2 vaccine epitopes specifically designed to elicit CD8 and/or CD4 TRM at sites of HSV-2 infection. This aspect of the disclosure is important because the usual sample for human CD4 and CD8 T cell research is blood. Genital tract HSV-2-specific CD4 and CD8 TRM cells could differ from blood HSV-specific CD8 T cells with regards to the epitopes that they recognize, or in other ways. Thus, the present disclosure concerns the fine specificity of HSV-2-specific CD8 and CD4 TRM active in the uterine cervix and/or in the genital skin at sites of healed, previous HSV-2 infection. Zhu et al. (2007) Journal of Experimental Medicine 204(3): 595-603.

For purposes of the present disclosure, epitopes are portions of viruses recognized by TRM T cell receptors (TCR) after the peptide is also physically bound by a human leukocyte antigen (HLA) molecule. An epitope includes specific amino acids that contact the binding portions of an HLA molecule and a TCR. Epitope determinants can include chemically active surface groupings of molecules such as amino acids, sugar side chains, phosphoryl or sulfonyl groups, and can have specific three-dimensional structural characteristics, and/or specific charge characteristics.

An “epitope” includes any determinant capable of being bound by an antigen-binding protein, such as a TCR. An epitope is a region of molecule that is bound by a binding protein that targets that region of molecule, and when that region of molecule is a protein, includes specific residues that directly contact the binding protein. In particular embodiments, an “epitope” denotes the binding site on a protein target bound by a corresponding binding domain. The binding domain either binds to a linear epitope, (e.g., an epitope including a stretch of 5 to 12 consecutive amino acids), or the binding domain binds to a three-dimensional structure formed by the spatial arrangement of several short stretches of the protein target. Three-dimensional epitopes recognized by a binding domain, e.g., by the epitope recognition site of a TCR or TCR fragment, can be thought of as three-dimensional surface features of an epitope molecule. These features fit precisely (in)to the corresponding binding site of the binding domain and thereby binding between the binding domain and its target protein is facilitated. In particular embodiments, an epitope can be considered to have two levels: (i) the “covered patch” which can be thought of as the shadow a TCR or binding domain would cast; and (ii) the individual participating side chains and backbone residues. Binding is then due to the aggregate of ionic interactions, hydrogen bonds, and hydrophobic interactions. Other portions of the epitope are thought to “face down” and bind to HLA molecules on the surfaces of infected cells or other antigen presenting cells.

CD8 T cells recognize small linear peptides in proteins, typically 8-20 amino acids long. This disclosure provides specific regions of HSV-2 proteins as vaccine epitopes. In particular embodiments, the vaccine epitopes elicit CD8 T cell responses in humans (e.g., infected humans). CD4 T cells also recognize similar linear peptides.

Biopsies of cervix or skin at sites of healed genital HSV-2 infection were digested to produce single cells. The cells were stimulated with whole HSV-2 to specifically activate any HSV-2-specific cells. The cells were stained with fluorescent antibodies to detect a surface protein that is up-regulated on activated cells. The fluorescent (+) CD8 T cells or CD4 (+) T cells were separately sorted and expanded in number. Preliminary assays confirmed that these bulk, polyclonal CD8 or CD4 T cell mixtures recognized HSV-2 whole virus. Other preliminary assays using technologies detailed in PMID 22214845, 23966859, 24554666, and 26810224 were performed to determine which full length HSV-2 genes and which HLA allelic variants were used by subpopulations within the bulk polyclonal CD8 T cells. Finally, synthetic peptides disclosed in Tables 1 and 2 below were tested with the bulk, polyclonal HSV-2-specific T cells to determine which peptides could activate the T cell mixture. At each stage, very strong, specific, and duplicate to triplicate signals with very low background were obtained leading to a high degree of confidence in the results.

Epitope-specific CD8 T cells bind to a combination of a peptide epitope and a cell surface molecule on the antigen presenting cell termed an HLA molecule. HLA molecules are encoded by several loci in the human genome and each has allelic variants. HLA class I molecules or antigens (HLA-A, HLA-B and HLA-C) are transmembrane proteins that are expressed on the surface of almost all the cells of the body (except for red blood cells and the cells of the central nervous system in the absence of inflammation) and present peptides on the cell surface, which peptides are produced from digested proteins that are broken down in the proteasomes.

Disclosed herein are the HSV-2 open reading frame as detailed in Genbank from HSV-2 strain HG52 (GenBank accession no. Z86099), the amino acid sequence of the reactive peptides that scored positive. The HLA alleles that are required for CD8 T cell recognition of these peptides are also described. For example, HLA A*0201 refers to the 0201 variant at the HLA A locus.

Particular HSV-2 vaccine epitopes disclosed herein include:

TABLE 1
CD8 TRM Epitopes (all sequences are HSV-2
strain 186 available as Genbank JX112656.1)

| HSV-2 Gene/ | Peptide | | HLA |
|---|---|---|---|
| Common Name | No. | Peptide Sequence | Allele |
| UL6 | 225-234 | AEYDRVHIYY | B*4402 |
| | | (SEQ ID NO: 1) | |
| UL23 thymidine | 360-369 | AEIRDLARTF | B*4402 |
| kinase | | (SEQ ID NO: 2) | |
| UL23 thymidine | 360-372 | AEIRDLARTFARE | B*4402 |
| kinase | | (SEQ ID NO: 3) | |
| UL19 major | 509-523 | WRQRLAHGRVRVVVAE | A*3201 |
| capsid VP5 | | (SEQ ID NO: 4) | |
| UL19 major | 1357-1371 | HFTQYLIYDASPLKG | A*0301 |
| capsid VPS | | (SEQ ID NO: 5) | |
| UL19 major | 1361-1374 | YLIYDASPLKGLSL | A*0301 |
| capsid VPS | | (SEQ ID NO: 6) | |
| UL49 VP22 | 45-57 | PMRARPRGEVRFL | B*0702 |
| tegument | | (SEQ ID NO: 7) | |
| RS1 ICP4 | 1123-1136 | ARLYPDAPPLRLCR | A*0301 |
| | | (SEQ ID NO: 8) | |
| RS1 ICP4 | 1123-1133 | ARLYPDAPPLR | A*0301 |
| | | (SEQ ID NO: 9) | |
| UL25 | 191-200 | TIADFPLTTR | A*6801 |
| | | (SEQ ID NO: 10) | |
| UL46 VP11/12 | 354-362 | ASDSLNNEY | A*0101 |
| tegument | | (SEQ ID NO: 11) | |
| UL46 VP11/12 | 249-263 | RLGPADRRFVALSGS | B*0702 |
| tegument | | (SEQ ID NO: 12) | |

In particular embodiments, the peptide epitopes include at least one epitope identified herein as an HLA B*4402-restricted epitope, at least one epitope identified herein as an HLA A*3201-restricted epitope, at least one epitope identified herein as an HLA A*6801-restricted epitope, at least one epitope identified herein as an HLA A*0201-restricted epitope, at least one epitope identified herein as an HLA B*0702-restricted epitope, and, optionally, at least one epitope identified herein as an HLA A*0101-restricted, and, optionally, at least one epitope identified herein as an HLA A*0301-restricted epitope. These HLA restricting alleles are desirable as they are prevalent genetic variants that are present at allele frequencies of 10-50% in many ethnically diverse populations. For example, between 30-60% of persons from many ethnicities have the HLA A*0201 allele. The HLA allelic variants B*0702, A*0101, and A*0301 are each present in 10-20% of people in several major ethnic groups. By using HLA based prioritization and combining these epitopes or regions that contain these epitopes in combination, one can create a vaccine candidate that would be expected to induce or boost CD8 responses in a large proportion of individuals in diverse ethnic groups.

CD4 TRM epitopes are also described. CD4 specific TRM epitopes include:

TABLE 2
CD4 TRM Epitopes (all sequences are HSV-2
strain 186 available as Genbank JX112656.1)

| HSV-2 Gene/ | Peptide | Peptide |
|---|---|---|
| Common Name | No. | Sequence |
| US6 glycoprotein D | 201-215 | ITQFILEHRARASCK |
| | | (SEQ ID NO: 13) |
| UL19 capsid VP5 | 305-319 | TYGEMVLNGANLVTA |
| | | (SEQ ID NO: 14) |
| UL49 VP22 tegument | 45-57 | PMRARPRGEVRFL |
| | | (SEQ ID NO: 15) |
| UL49 VP22 tegument | 49-61 | RPRGEVRFLHYDE |
| | | (SEQ ID NO: 16) |
| UL27 glycoprotein B | 693-707 | DSGLLDYTEVQRRNQ |
| | | (SEQ ID NO: 17) |
| UL27 glycoprotein B | 697-711 | LDYTEVQRRNQLHDL |
| | | (SEQ ID NO: 18) |
| UL29 DNA binding protein | 813-827 | GPLGFLLKQFHAVIF |
| | | (SEQ ID NO: 19) |
| UL29 DNA binding protein | 817-831 | FLLKQFHAVIFPNGK |
| | | (SEQ ID NO: 20) |
| UL29 DNA binding protein | 865-879 | IAFIKRFSLDYGAIN |
| | | (SEQ ID NO: 21) |
| UL29 DNA binding protein | 869-883 | KRFSLDYGAINFINL |
| | | (SEQ ID NO: 22) |

As indicated, in particular embodiments, the HSV-2 vaccine includes a subunit vaccine. A subunit vaccine can refer to a vaccine that does not contain a whole live or killed pathogen, but only a subunit (e.g., a single protein or protein fragment) of the pathogen that stimulates an immune response against the pathogen.

In particular embodiments, the HSV-2 vaccines are referred to as HSV-2 therapeutics and can include immunogenic proteins. An immunogenic protein can, for example, be used to elicit a TRM response in a subject.

In particular embodiments, HSV-2 therapeutics (e.g., HSV-2 vaccines, immunogenic proteins) can include multimerization domains. Multimerization domains can allow for multimerization of the HSV-2 vaccine proteins, which can enhance their immunogenicity. In particular embodiments, the multimerization domain is C4b multimerization domain. C4 binding protein (C4b) is the major inhibitor of the classical complement and lectin pathway. The complement system is a major part of innate immunity and is the first line of defense against invading microorganisms. Orchestrated by more than 60 proteins, its major task is to discriminate between host cells and pathogens and to initiate immune responses when necessary. It also recognizes necrotic or apoptotic cells. Hofmeyer et al., J Mol Biol. 2013 Apr 26;425(8):1302-17.

Full-length native C4b includes seven α-chains linked together by a multimerization (i.e., heptamerization) domain at the C-terminus of the α-chains. Blom et al., (2004) Mol Immunol 40: 1333-1346. One of the α-chains can be replaced by a β-chain in humans. The wild-type C4b multimerization domain is 57 amino acid residues in humans and 54 amino acid residues in mice. Forbes et al., PLoS One. 2012; 7(9): e44943. It contains an amphipathic α-helix region, which is necessary and sufficient for heptamerization, as well as two cysteine residues which stabilize the structure. Kask et al., (2002) Biochemistry 41: 9349-9357.

Examples of C4b multimerization domains are provided in Table 3.

TABLE 3
Exemplary C4b Multimerization Domains

| SEQ ID | |
|---|---|
| NO. | Sequence |
| 23 | SGRAHAGWETPEGCEQVLTGKRLMQCLPNPEDVKMALEVYKL |
| | SLEIEQLELQRDSARQSTLDKELVPR |
| 24 | KKQGDADVCGEVAYIQSVVSDCHVPTAELRTLLEIRKLFLEI |
| | QKLKVELQGLSKE |
| 25 | ETPEGCEQVLTGKRLMQCLPNPEDVKMALEVYKLSLEIEQLE |
| | LQRDSARQSTLDKEL |
| 26 | WETPEGCEQVLTGKRLMQCLPNPEDVKMALEVYKLSLEIEQL |
| | ELQRDSARQSTLDKEL |
| 27 | CEQVLTGKRLMQCLPNPEDVKMALEVYKLSLEIEQLELQRDS |
| | ARQSTLDKEL |

In particular embodiments, the C4b multimerization domain will be a multimerization domain which includes (i) glycine at position 12, (ii) alanine at position 28, (iii) leucines at positions 29, 34, 36, and/or 41; (iv) tyrosine at position 32; (v) lysine at position 33; and/or (vi) cysteine at positions 6 and 18. In particular embodiments, the C4b multimerization domain will be a multimerization domain which includes (i) glycine at position 12, (ii) alanine at position 28, (iii) leucines at positions 29, 34, 36, and 41; (iv) tyrosine at position 32; (v) lysine at position 33; and (vi) cysteine at positions 6 and 18.

C4b multimerization domains can include any of SEQ ID NOs: 23-27 with an N-terminal deletion of at least 1 consecutive amino acid residues (e.g. at least 2, 3, 4, 5, 6, 7, 8, 9, 10 consecutive amino acid residues) in length. Additional embodiments can include a C-terminal deletion of at least 1 consecutive amino acid residues (egg. at least 2, 3, 4, 5, 6, 7, 8, 9, 10 consecutive amino acid residues) in length.

Particular C4b multimerization domain embodiments will retain or will be modified to include at least 1 of the following residues: A6; E11; A13; D21; C22; P25; A27; E28; L29; R30; T31; L32; L33; E34; I35; K37; L38; L40; E41; 142; Q43; K44; L45; E48; L49; or Q50. Further embodiments will retain or will be modified to include A6; E11; A13; D21; C22; P25; A27; E28; L29; R30; T31; L32; L33; E34; I35; K37; L38; L40; E41; I42; Q43; K44; L45; E48; L49; and Q50. Particular C4b multimerization domain embodiments will include the amino acid sequence “AELR”.

In particular embodiments, HSV-2 therapeutics have high affinity for CD8 or CD4 TRM as evidenced by binding between the therapeutic and the TRM. In particular embodiments, an “HSV-2-reactive” CD8 or CD4 T cell has high binding affinity for an HSV-2 therapeutic. In particular embodiments, a CD8 or CD4 T cell that binds an HSV-2 therapeutic is “reactive to” that HSV-2 therapeutic. In particular embodiments “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., TCR and epitope). The affinity of a molecule X for its partner Y can generally be represented by the dissociation constant (K_{D}) or the association constant (K_{A}). Affinity can be measured by common methods known in the art.

In particular embodiments, “bind” means that the binding domain of the TCR associates with its target epitope with a dissociation constant (K_{D}) of 10⁻⁸ M or less, in particular embodiments of from 10⁻⁵ M to 10⁻¹³ M, in particular embodiments of from 10⁻⁵ M to 10⁻¹⁰ M, in particular embodiments of from 10⁻⁵ M to 10⁻⁷ M, in particular embodiments of from 10⁻⁸ M to 10⁻¹³ M, or in particular embodiments of from 10⁻⁹ M to 10⁻¹³ M. The term can be further used to indicate that the binding domain does not bind to other biomolecules present, (e.g., it binds to other biomolecules with a dissociation constant (K_{D}) of 10⁻⁴ M or more, in particular embodiments of from 10⁻⁴ M to 1 M).

In particular embodiments, “bind” means that the binding domain of the TCR associates with its target epitope with an affinity constant (i.e., association constant, K_{A}) of 10⁷ M⁻¹ or more, in particular embodiments of from 10⁵ M⁻¹ to 10¹³ M⁻¹, in particular embodiments of from 10⁵ M⁻¹ to 10¹⁰ M⁻¹, in particular embodiments of from 10⁵ M⁻¹ to 10⁸ M⁻¹, in particular embodiments of from 10⁷ M⁻¹ to 10¹³ M⁻¹, or in particular embodiments of from 10⁷ M⁻¹ to 10⁸ M⁻¹. The term can be further used to indicate that the binding domain does not bind to other biomolecules present, (e.g., it binds to other biomolecules with an association constant (K_{A}) of 10⁴ M⁻¹ or less, in particular embodiments of from 10⁴ M⁻¹ to 1 M⁻¹).

As indicated previously, variants of the sequences disclosed and referenced herein are included. In particular embodiments, variants of proteins can include those having one or more conservative amino acid substitutions or one or more non-conservative substitutions that do not adversely affect the function of the protein. A “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gln); Group 4: Arginine (Arg), Lysine (Lys), Histidine (His); Group 5: Isoleucine (Ile), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

Those skilled in the art are aware that the HSV epitope can still be immunologically effective with a small portion of adjacent HSV or other amino acid sequence present.

A fragment of a polypeptide consists of less than the complete amino acid sequence of the corresponding protein, but includes the recited epitope or antigenic region. As is understood in the art and confirmed by assays conducted using fragments of widely varying lengths, additional sequence beyond the recited epitope can be included without hindering the immunological response. A fragment of the invention can be as few as 8 amino acids in length, or can encompass 10%, 20%, 30%, 40%, 50%, 60%, 70%, 80%, 90% or 95% of the full length of the protein.

In particular embodiments, the polypeptide is fused with or co-administered with a heterologous peptide. The heterologous peptide can be another epitope or an unrelated sequence. The unrelated sequence may be inert or it may facilitate the immune response. In typical embodiments, the epitope is part of a multi-epitopic vaccine, in which numerous epitopes are combined in one polypeptide.

The epitope can be part of a fusion protein. In particular embodiments, the fusion protein is soluble. A soluble fusion protein can be suitable for injection into a subject and for eliciting an immune response. Within particular embodiments, a polypeptide can be a fusion protein that includes multiple epitopes as described herein, or that includes at least one epitope described herein and an unrelated sequence. In one example, the fusion protein includes a HSV epitope described herein (with or without flanking adjacent native sequence) fused with a non-native sequence. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., 1985, Gene 40:39-46; Murphy et al., 1986, Proc. Natl. Acad. Sci. USA 83:8258-8262; U.S. Pat. No. 4,935,233 and U.S. Pat. No. 4,751 ,180. The linker sequence may generally be from 1 to 50 amino acids in length. Linker sequences are not required when the first and second polypeptides have non-essential N-terminal amino acid regions that can be used to separate the functional domains and reduce or prevent steric interference. Particular embodiments can utilize Gly-Ser linkers.

In particular embodiments, variants of the protein sequences (e.g., antibodies, vaccine proteins, and/or multimerization domains) disclosed herein include sequences with at least 70% sequence identity, at least 80%, at least 85%, at least 90%, at least 95%, at least 96%, at least 97%, at least 98%, or at least 99% sequence identity to the protein sequences described or disclosed herein.

“% sequence identity” refers to a relationship between two or more sequences, as determined by comparing the sequences. In the art, “identity” also means the degree of sequence relatedness between protein sequences or nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, N.J. (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine % sequence identity are designed to give the best match between the sequences tested. Methods to determine % sequence identity and similarity are codified in publicly available computer programs. Sequence alignments and % sequence identity calculations may be performed using the Megalign program of the LASERGENE bioinformatics computing suite (DNASTAR, Inc., Madison, Wisc.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisc.); BLASTP, BLASTN, BLASTX (Altschul, et al., J. Mol. Biol. 215:403-410 (1990); DNASTAR (DNASTAR, Inc., Madison, Wisc.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

In particular embodiments, variants have been modified from a reference sequence to produce an administration benefit. Exemplary administration benefits can include (1) reduced susceptibility to proteolysis, (2) reduced susceptibility to oxidation, (3) altered binding affinity for forming protein complexes, (4) altered binding affinities, (5) reduced off-target immunogenicity; and/or (6) extended half-life.

In particular embodiments, modified HSV-2 therapeutics include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications also include nitrited proteins.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, A. et al., Cancer Biochem. Biophys.,1984, 7:175-186; Hershfield, M. S. et al., N. Engl. J. Medicine, 1987, 316:589-596; and Meyers, F. J. et al., Clin. Pharmacol. Ther., 1991, 49:307-313). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta vol. 788, pg. 248 (1984)), alter protein immunogenicity (Abuchowski et al.; J. Biol. Chem. vol. 252 pg. 3582 (1977)), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

In particular embodiments, HSV-2 therapeutics can be linked to human serum albumin (HSA). Linkage to HSA can increase the size of the protein and can increase serum half-life. An HSA-linkage can increase HSV-2 therapeutic half-life without altering the binding and/or activity of the HSV-2 therapeutic.

One can readily confirm the suitability of a particular variant by assaying the ability of the variant polypeptide to elicit an immune response. The ability of the variant to elicit an immune response can be compared to the response elicited by the parent polypeptide assayed under experimentally comparable control conditions. One example of an immune response is a cellular immune response. The assaying can include performing an assay that measures T cell stimulation or activation. Examples of T cells include CD4 and CD8 T cells.

One example of a T cell stimulation assay is a cytotoxicity assay, such as that described in Koelle, DM et al., Human Immunol. 1997, 53;195-205. In one example, the cytotoxicity assay includes contacting a cell that presents the antigenic viral peptide in the context of the appropriate HLA molecule with a T cell, and detecting the ability of the T cell to kill the antigen presenting cell. Cell killing can be detected by measuring the release of radioactive ⁵¹Cr from the antigen presenting cell. Release of ⁵¹Cr into the medium from the antigen presenting cell is indicative of cell killing. An exemplary criterion for increased killing is a statistically significant increase in counts per minute (cpm) based on counting of ⁵¹Cr radiation in media collected from antigen presenting cells admixed with T cells as compared to control media collected from antigen presenting cells admixed with media. In particular embodiments, an “HSV-2-reactive” CD8 or CD4 T cell has increased cell killing as compared to CD8 or CD4 T cells in control media in a cytotoxicity assay presenting an HSV-2 therapeutic on an antigen presenting cell. In particular embodiments, a CD8 or CD4 T cell having increased cell killing as compared to CD8 or CD4 T cells in control media in a cytotoxicity assay presenting an HSV-2 therapeutic is “reactive to” that HSV-2 therapeutic.

In particular embodiments, an “HSV-2-reactive” CD8 or CD4 T cell has increased expression of CD137 activation marker in T cell functional assays described herein compared to CD8 or CD4 T cells that have been treated under control conditions. In particular embodiments, a CD8 or CD4 T cell having increased expression of CD137 activation marker in T cell functional assays described herein compared to CD8 or CD4 T cells that have been treated under control conditions is “reactive to” that HSV-2 therapeutic. In particular embodiments, an “HSV-2-reactive” CD8 or CD4 T cell has increased production of IFN-γ or IL-2 in T cell functional assays described herein compared to CD8 or CD4 T cells that have been treated under control conditions. In particular embodiments, a CD8 or CD4 T cell having increased production of IFN-γ or IL-2 in T cell functional assays described herein compared to CD8 or CD4 T cells that have been treated under control conditions is “reactive to” that HSV-2 therapeutic. In particular embodiments, an “HSV-2-reactive” CD8 or CD4 T cell has increased proliferation in T cell functional assays described herein compared to CD8 or CD4 T cells that have been treated under control conditions.

In particular embodiments, a CD8 or CD4 T cell having increased proliferation in T cell functional assays described herein compared to CD8 or CD4 T cells that have been treated under control conditions is “reactive to” that HSV-2 therapeutic.

In particular embodiments, the HSV-2 therapeutics are produced from a gene using a protein expression system. Protein expression systems can utilize DNA constructs (e.g., chimeric genes, expression cassettes, expression vectors, recombination vectors) including a nucleic acid sequence encoding the protein or proteins of interest operatively linked to appropriate regulatory sequences. In particular embodiments, such DNA constructs are not naturally-occurring DNA molecules and are useful for introducing DNA into host-cells to express selected proteins of interest. In particular embodiments, a DNA construct that encodes an HSV-2 therapeutic can be inserted into cells (e.g., bacterial, mammalian, insect, etc.), which can produce the HSV-2 therapeutic encoded by the DNA construct.

Operatively linked refers to the linking of DNA sequences (including the order of the sequences, the orientation of the sequences, and the relative spacing of the various sequences) in such a manner that the encoded protein is expressed. Methods of operatively linking expression control sequences to coding sequences are well known in the art. See, e.g., Maniatis et al., Molecular Cloning: A Laboratory Manual, Cold Spring Harbor, N. Y., 1982; and Sambrook et al., Molecular Cloning: A Laboratory Manual, Cold Spring Harbor, N. Y., 1989.

Expression control sequences are DNA sequences involved in any way in the control of transcription or translation. Suitable expression control sequences and methods of making and using them are well known in the art. Expression control sequences generally include a promoter. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds, Nucleic Acids Res., 15, 2343-2361, 1987. Also, the location of the promoter relative to the transcription start may be optimized. See, e.g., Roberts et al., Proc. Natl. Acad. Sci. USA, 76:760-764, 1979.

The promoter may include, or be modified to include, one or more enhancer elements. In particular embodiments, the promoter will include a plurality of enhancer elements. Promoters including enhancer elements can provide for higher levels of transcription as compared to promoters that do not include them.

For efficient expression, the coding sequences can be operatively linked to a 3′ untranslated sequence. In particular embodiments, the 3′ untranslated sequence can include a transcription termination sequence and a polyadenylation sequence. The 3′ untranslated region can be obtained, for example, from the flanking regions of genes.

In particular embodiments, a 5′ untranslated leader sequence can also be employed. The 5′ untranslated leader sequence is the portion of an mRNA that extends from the 5′ CAP site to the translation initiation codon.

In particular embodiments, a “hisavi” tag can be added to the N-terminus or C-terminus of a gene by the addition of nucleotides coding for the Avitag amino acid sequence, “GLNDIFEAQKIEWHE” (SEQ ID NO: 29), as well as the 6xhistidine tag coding sequence “HHHHHH (SEQ ID NO: 28)”. The Avitag avidity tag can be biotinylated by a biotin ligase to allow for biotin-avidin or biotin-streptavidin based interactions for protein purification, as well as for immunobiology (such as immunoblotting or immunofluorescence) using anti-biotin antibodies. The 6xhistidine tag allows for protein purification using Ni-2+ affinity chromatography.

In particular embodiments, HSV-2 therapeutics disclosed herein can be produced using the Daedalus expression system as described in Pechman et al., Am J Physiol 294: R1234-R1239, 2008. The Daedalus system utilizes inclusion of minimized ubiquitous chromatin opening elements in transduction vectors to reduce or prevent genomic silencing and to help maintain the stability of decigram levels of expression. This system can bypass tedious and time-consuming steps of other protein production methods by employing the secretion pathway of serum-free adapted human suspension cell lines, such as 293 Freestyle. Using optimized lentiviral vectors, yields of 20-100 mg/l of correctly folded and post-translationally modified, endotoxin-free protein of up to 70 kDa in size, can be achieved in conventional, small-scale (100 ml) culture. At these yields, most proteins can be purified using a single size-exclusion chromatography step, immediately appropriate for use in structural, biophysical or therapeutic applications. Bandaranayake et al., Nucleic Acids Res., 2011 (Nov); 39(21). In some instances, purification by chromatography may not be needed due to the purity of manufacture according the methods described herein.

In particular embodiments, the DNA constructs can be introduced by transfection, a technique that involves introduction of foreign DNA into the nucleus of eukaryotic cells. In particular embodiments, the proteins can be synthesized by transient transfection (DNA does not integrate with the genome of the eukaryotic cells, but the genes are expressed for 24-96 hours). Various methods can be used to introduce the foreign DNA into the host-cells, and transfection can be achieved by chemical-based means including by the calcium phosphate, by dendrimers, by liposomes, and by the use of cationic polymers. Non-chemical methods of transfection include electroporation, sonoporation, optical transfection, protoplast fusion, impalefection, and hydrodynamic delivery. In particular embodiments, transfection can be achieved by particle-based methods including gene gun where the DNA construct is coupled to a nanoparticle of an inert solid which is then “shot” directly into the target-cell's nucleus. Other particle-based transfection methods include magnet assisted transfection and impalefection.

Nucleic acid sequences encoding proteins disclosed herein can be derived by those of ordinary skill in the art. Nucleic acid sequences can also include one or more of various sequence polymorphisms, mutations, and/or sequence variants (e.g., splice variants or codon optimized variants). In particular embodiments, the sequence polymorphisms, mutations, and/or sequence variants do not affect the function of the encoded protein.

Sequence information provided by public databases can be used to identify additional gene and protein sequences that can be used within the teachings of the current disclosure.

In particular embodiments, T cells including T cell receptors (TCRs) that bind HSV-2 epitopes are used in adoptive T cell transfer into a subject to treat HSV-2 infection. Adoptive cell transfer (ACT) is the passive transfer of ex vivo grown cells, most commonly immune-derived cells, into a host with the goal of transferring the immunologic functionality and characteristics of the transplant. In the present disclosure, isolated and expanded HSV-2-reactive CDS and/or HSV-2-reactive CD4 T cells can be transferred into a subject infected with HSV-2 to treat the HSV-2 infection or into a non-infected subject to reduce or prevent HSV-2 infection, In particular embodiments, the isolated and expanded CD8 and/or CD4 HSV-2-reactive T cells include HSV-2-reactive CDS and/or HSV-2-reactive CD4 TR M cells. In particular embodiments, the isolated and expanded HSV-2-reactive CD8 and/or HSV-2-reactive CD4 T cells include CD137 high cells,

Adoptive cell transfer can be autologous, as is common in adoptive T-cell therapies, or allogeneic, as typical for treatment of infections or graft-versus-host disease. In particular embodiments, the adoptive T cell therapy including isolated and expanded HSV-2-reactive CD8 and/or HSV-2-reactive CD4 T cells, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing, are administered to the same subject. In particular embodiments, the adoptive T cell therapy including isolated and expanded HSV-2-reactive CD8 and/or HSV-2-reactive CD4 T cells, is carried out by allogeneic transfer, in which the cells are isolated and/or otherwise prepared from a subject other than a subject who is to receive or who ultimately receives the cell therapy, e.g., a first subject. In particular embodiments, the cells then are administered to a different subject, e.g., a second subject, of the same species. In particular embodiments, the first and second subjects are genetically identical or similar. In particular embodiments, the second subject expresses the same HLA class or supertype as the first subject.

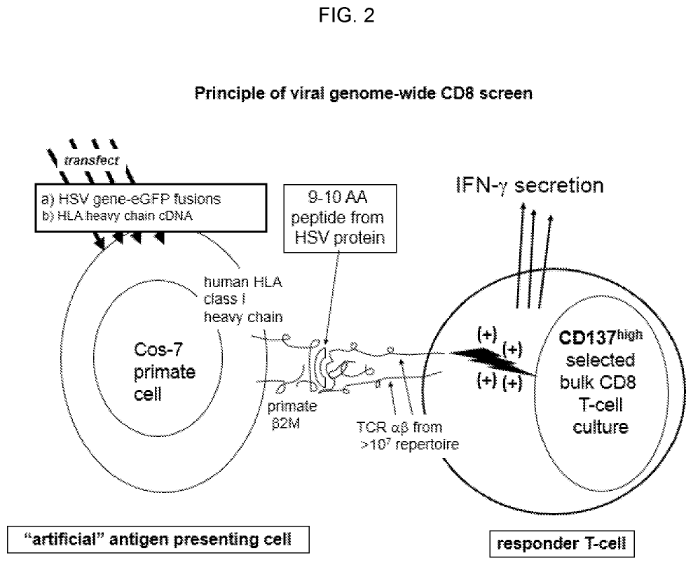

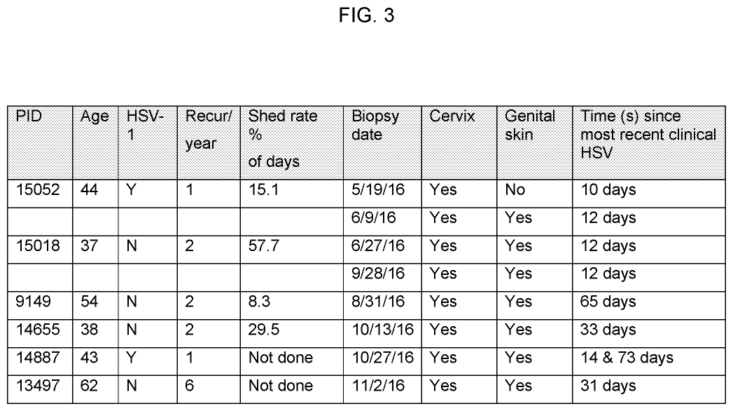

The isolated and expanded HSV-2-reactive CD8 and/or HSV-2-reactive CD4 T cells to be used in ACT can be identified and obtained using the methods described herein. In particular embodiments, HSV-2-reactive CD8 and/or HSV-2-reactive CD4 T cells can be obtained from skin and/or cervical biopsies. In particular embodiments, HSV-2-reactive CD8 and/or HSV-2-reactive CD4 T cells can be identified by genome-wide or proteome-wide CDS and/or CD4 screens as described in FIGS. 2. 9, and 13. In particular embodiments, HSV-2-reactive CD8 and/or HSV-2-reactive CD4 T cells can initially be selected based on high expression of T cell activation marker CD137. In particular embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

Expansion of HSV-2-reactive CD8 and/or HSV-2-reactive CD4 T cells for adoptive cell transfer can be accomplished by any number of methods as are known in the art. Cell culture and/or expansion conditions can include one or more of particular media, temperature, oxygen content, carbon dioxide content, time, agents, e.g., nutrients, amino acids, antibiotics, anti-viral compounds, ions, mitogens, and/or stimulatory factors, such as cytokines, chemokines, antigens, binding partners, fusion proteins, recombinant soluble receptors, and any agents designed to activate the cells. In particular embodiments, expansion of the T cells is carried out in the presence of an agent capable of activating one or more intracellular signaling domains of one or more components of a TCR complex, such as a CD3 zeta chain, or capable of activating signaling through such a complex or component. Such agents can include antibodies, such as those specific for a TCR component and/or costimulatory receptor, e.g., anti-CD3, anti-CD28, anti-4-1BB, bound to a solid support such as a bead, and/or one or more cytokines. Optionally, the expansion method may further include the step of adding anti-CD3 and/or anti-CD28 antibody to the culture medium.

The expansion method may further include expansion in the presence of Interleukin (IL)-2, IL-15, IL-7, and/or IL-21. In particular embodiments, the aforementioned cytokines can be added to the culture medium.

In particular embodiments, the CDS and/or CD4 cell populations are expanded by adding feeder cells, such as non-dividing peripheral blood mononuclear cells (PBMC), (e.g., such that the resulting population of cells contains at least 5, 10, 20, or 40 or more PBMC feeder cells for each T cell in the initial population to be expanded); and incubating the culture (e.g. for a time sufficient to expand the numbers of T cells). In particular embodiments, the non-dividing feeder cells can include gamma-irradiated PBMC feeder cells. In particular embodiments, the feeder cells are added to culture medium prior to the addition of the populations of T cells.

In particular embodiments, expansion conditions include temperature suitable for the growth of T cells, for example, at least 25° C., at least 30° C., and generally at 37° C. In particular embodiments, a temperature shift is effected during culture, such as from 37° C. to 35° C. Optionally, the expansion may further include adding non-dividing EBV-transformed lymphoblastoid cells (LCL) as feeder cells. LCL can be irradiated with gamma rays. The LCL feeder cells in particular embodiments are provided in any suitable amount, such as a ratio of LCL feeder cells to initial T cells of at least 10:1.

In particular embodiments, the T cells can be expanded by re-stimulation with an HSV-2 protein or protein fragment pulsed onto HLA-expressing antigen-presenting cells. In particular embodiments, the T cells can be re-stimulated with irradiated, autologous lymphocytes or with irradiated HLA-expressing allogeneic lymphocytes and IL-2.

In particular embodiments, the HSV-2-reactive CDB and/or HSV-2-reactive CD4 T cells can be frozen, e.g., cryopreserved, either before or after isolation and/or expansion. In particular embodiments, the cells are suspended in a freezing solution, e.g., following a washing step to remove plasma and platelets. Any of a variety of known freezing solutions and parameters may be used, One example involves using PBS containing 20% DMSO and 8% human serum albumin (HSA), or other suitable cell freezing media. This is then diluted 1:1 with media so that the final concentration of DMSOl and HSA are 10% and 4%, respectively. The cells are then frozen to 80° C. at a rate of 1° per minute and stored in the vapor phase of a liquid nitrogen storage tank.

In particular embodiments, expansion is carried out in accordance with techniques such as those described in U.S. Pat. No. 6,040,177 to Riddell et al., Kiebanoff et al. (2012) J Immunother. 35(9): 651-660, Terakura et al. (2012) Blood. 1:72-82, and/or Wang et al. (2012) J Immunother. 3(9):689-701.

HSV-2 therapeutics can be formulated alone or in combination into compositions for administration to subjects. Salts and/or pro-drugs of HSV-2 therapeutics can also be used.

A pharmaceutically acceptable salt includes any salt that retains the activity of the HSV-2 therapeutic and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage of an HSV-2 therapeutic or by hydrolysis of a biologically labile group.

In particular embodiments, compositions disclosed herein include an HSV-2 therapeutic of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the HSV-2 therapeutic or helps to reduce or prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous administration and more particularly by intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid formulations such as powders, capsules and tablets, suitable excipients include binders (gum tragacanth, acacia, cornstarch, gelatin), fillers such as sugars, e.g.,lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of HSV-2 therapeutic composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as sparingly soluble salts.

Additionally, compositions can be formulated as sustained-release systems utilizing semipermeable matrices of solid polymers including at least one HSV-2 therapeutic. Various sustained-release materials have been established and are well known by those of ordinary skill in the art. Sustained-release systems may, depending on their chemical nature, release one or more HSV-2 therapeutics following administration for a few weeks up to over 100 days. Depot preparations can be administered by injection; parenteral injection; instillation; or implantation into soft tissues, a body cavity, or occasionally into a blood vessel with injection through fine needles.

Depot formulations can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Del.), poly(vinyl alcohol) (PVA), Brij® (Croda Americas, Wilmington, Del.), sucrose acetate isobutyrate (SAIB), salts, and buffers.

Excipients that partition into the external phase boundary of microparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot formulations can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

HSV-2 therapeutics can be formulated in combination with one or more adjuvants into compositions for administration to subjects. The term “adjuvant” refers to material that enhances the immune response to a vaccine antigen and is used herein in the customary use of the term. The precise mode of action is not understood for all adjuvants, but such lack of understanding does not prevent their clinical use for a wide variety of vaccines.

In particular embodiments, an adjuvant that can be used include an adjuvant that elicits TRM. In particular embodiments, an adjuvant formulated with an HSV-2 vaccine can include saponins. Saponins are steroid or triterpenoid glycosides found in plants, lower marine animals and some bacteria. Saponins contain a steroidal or triterpenoid aglycone to which one or more sugar chains are attached. Triterpenoid saponins from the bark of the Quillaja saponaria tree are potent adjuvants. Quil A is a saponin fraction derived from an aqueous extract from the Quillaja tree bark, and the structure of QS-21, a fraction purified from this extract, is shown below:

Structure of QS-21 from Quillaja Saponaria Molina

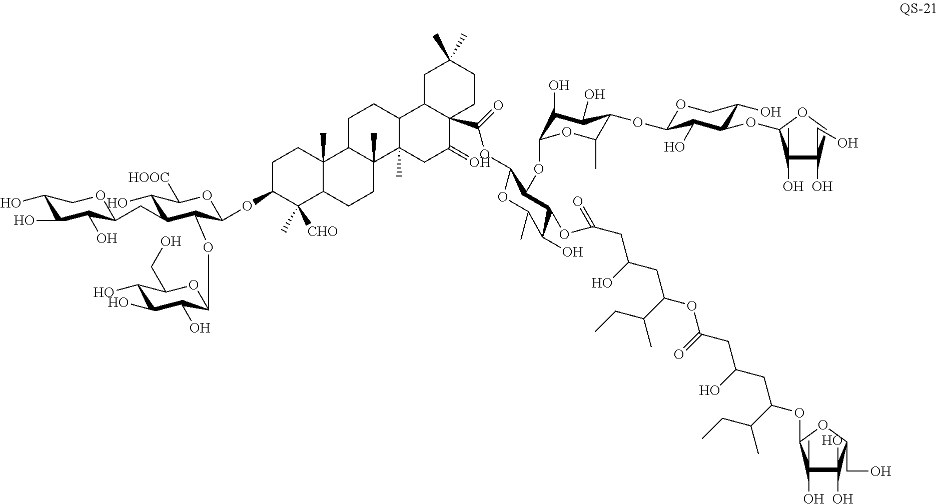

Quillaja saponins (QS) include a heterogeneous mixture of related but different chemical structures with various immunostimulatory activities, safety profiles, and particle forming properties. Saponin-based adjuvants can be formulated in free form, with aluminum hydroxide, in an immunostimulating complex (ISCOM), or in ISCOM-Matrix/Matrix structures (Morein et al. (1984) Nature 308: 4577-460; Lovgren & Morein (1988) Biotechnology and Applied Biochemistry 10: 161-172). See also, for example, EP0436620; EP0109952; EP0109942; EP0180564; and EP0242380. In particular embodiments, an adjuvant formulated with an HSV-2 vaccine can include 40 nm nanoparticles including Quillaja saponins, cholesterol and phospholipid (Reimer et al. (2012) PLoS ONE 7(7): e41451). In particular embodiments, an adjuvant formulated with an HSV-2 vaccine can include a saponin-based adjuvant such as matrix-MTM (Novavax, Gaithersburg, Md.). In particular embodiments, an adjuvant formulated with an HSV-2 vaccine can include a saponin-based adjuvant such as matrix-M2™ (Novavax, Gaithersburg, Md.).

In particular embodiments, an adjuvant formulated with an HSV-2 vaccine can include a saponin-based adjuvant including QS-21, 3-deacylated monophosphoryl lipid (MPL), and liposomes (AS01 Adjuvant System). In particular embodiments, an adjuvant formulated with an HSV-2 vaccine can include a saponin-based adjuvant including QS-21, MPL, with an oil in water emulsion (AS02 Adjuvant System). In particular embodiments, an adjuvant formulated with an HSV-2 vaccine can include an oil in water emulsion with alpha-tocopherol (Vitamin E) as immuno-enhancing component (AS03 Adjuvant System). In particular embodiments, an adjuvant formulated with an HSV-2 vaccine can include MPL adsorbed onto aluminum hydroxide or aluminum phosphate (ASO4 Adjuvant System). In particular embodiments, an adjuvant formulated with an HSV-2 vaccine can include a saponin-based adjuvant including CpG 7909, QS-21 and MPL with liposomes (AS15 Adjuvant System). AS01, AS02, AS03, AS04, and AS15 Adjuvant Systems are from GSK Vaccines, Wavre, Belgium and described in Garcon and Pasquale 2017 Hum Vaccin Immunother 13(1): 19-33. In particular embodiments, an adjuvant formulated with an HSV-2 vaccine can include a TLR4 agonist glucopyranosyl lipid A (GLA) formulated in stable emulsion (GLA SE; Odegard et al. (2016) Vaccine 34(1): 101-109). In particular embodiments, an adjuvant formulated with an HSV-2 vaccine can include a carbomer-lecithin-based adjuvant (e.g., Adjuplex™, Millipore Sigma, Burlington, Mass.; Wegmann et al. (2015) Clin Vaccine Immunol. CVI-00736).

Additional exemplary vaccine adjuvants include any kind of Toll-like receptor ligand or combinations thereof (e.g. CpG, Cpg-28 (a TLR9 agonist), Polyriboinosinic polyribocytidylic acid (Poly(I:C)), α-galactoceramide, MPLA, Motolimod (VTX-2337, a novel TLR8 agonist developed by VentiRx), IMO-2055 (EMD1201081), TMX-101 (imiquimod), MGN1703 (a TLR9 agonist), G100 (a stabilized emulsion of the TLR4 agonist glucopyranosyl lipid A), Entolirnod (a derivative of Salmonella flagellin also known as CBLB502), Hiltonol (a TLR3 agonist), and Imiquimod), and/or inhibitors of heat-shock protein 90 (Hsp90), such as 17-DMAG (17-dimethylaminoethylamino-17-demethoxygeldanamycin).

In particular embodiments a squalene-based adjuvant can be used. Squalene is part of the group of molecules known as triterpenes, which are all hydrocarbons with 30 carbon molecules. Squalene can be derived from certain plant sources, such as rice bran, wheat germ, amaranth seeds, and olives, as well as from animal sources, such as shark liver oil. In particular embodiments, the squalene-based adjuvant is MF59® (Novartis, Basel, Switzerland). An example of a squalene-based adjuvant that is similar to MF59® but is designed for preclinical research use is Addavax™ (InvivoGen, San Diego, Calif.). MF59 has been FDA approved for use in an influenza vaccine, and studies indicate that it is safe for use during pregnancy (Tsai T, et al. Vaccine. 2010. 17:28(7):1877-80; Heikkinen T, et al. Am J Obstet Gynecol. 2012. 207(3):177). In particular embodiments, squalene based adjuvants can include 0.1% -20% (v/v) squalene oil. In particular embodiments, squalene based adjuvants can include 5%(v/v) squalene oil.

In particular embodiments the adjuvant alum can be used. Alum refers to a family of salts that contain two sulfate groups, a monovalent cation, and a trivalent metal, such as aluminum or chromium. Alum is an FDA approved adjuvant. In particular embodiments, vaccines can include alum in the amounts of 1-1000 μg/dose or 0.1 mg-10 mg/dose.

In particular embodiments, one or more STING agonists are used as a vaccine adjuvant. “STING” is an abbreviation of “stimulator of interferon genes”, which is also known as “endoplasmic reticulum interferon stimulator (ERIS)”, “mediator of IRF3 activation (MITA)”, “MPYS” or “transmembrane protein 173 (TM173)”.

In particular embodiments, STING agonists include cyclic molecules with one or two phosphodiester linkages, and/or one or two phosphorothioate diester linkages, between two nucleotides. This includes (3′,5′)-(3′,5′) nucleotide linkages (abbreviated as (3′,3′)); (3′,5′)-(2′,5′) nucleotide linkages (abbreviated as (3′,2′)); (2′,5′)-(3′,5′) nucleotide linkages (abbreviated as (2′,3′)); and (2′,5′)-(2′,5′) nucleotide linkages (abbreviated as (2′,2′)). “Nucleotide” refers to any nucleoside linked to a phosphate group at the 5′, 3′ or 2′ position of the sugar moiety.

In particular embodiments, STING agonists include c-AIMP; (3′,2′)c-AIMP; (2′,2′)c-AIMP; (2′,3′)c-AIMP; c-AIMP(S); c-(dAMP-dIMP); c-(dAMP-2′FdIMP); c-(2′FdAMP-2′FdIMP); (2′,3′)c-(AMP-2′FdIMP); c-[2′FdAMP(S)-2′FdIMP(S)]; c-[2′FdAMP(S)-2′FdIMP(S)](POM)2; and DMXAA. Additional examples of STING agonists are described in WO2016/145102.

Other immune stimulants can also be used as vaccine adjuvants. Additional exemplary small molecule immune stimulants include TGF-β inhibitors, SHP-inhibitors, STAT-3 inhibitors, and/or STAT-5 inhibitors. Exemplary siRNA capable of down-regulating immune-suppressive signals or oncogenic pathways (such as kras) can be used whereas any plasmid DNA (such as minicircle DNA) encoding immune-stimulatory proteins can also be used.

In particular embodiments, the immune stimulant may be a cytokine and or a combination of cytokines, such as IL-1β, IL-2, IL-12 or IL-15 in combination with IFN-α, IFN-β or IFN-γ, or GM-CSF, or any effective combination thereof, or any other effective combination of cytokines. The above-identified cytokines stimulate T_{H}1 responses, but cytokines that stimulate T_{H}2 responses may also be used, such as IL-4, IL-10, IL-11, or any effective combination thereof. Also, combinations of cytokines that stimulate T_{H}1 responses along with cytokines that stimulate T_{H}2 responses may be used.

Compositions of the present disclosure can include isolated, expanded and HSV-2-reactive CD8 and/or HSV-2-reactive CD4 T cells that can be used to treat, reduce, or prevent HSV-2 infection. In particular embodiments, a range of HSV-2-reactive CD8 and/or HSV-2-reactive CD4 T cells present in a composition can include from 1×10⁸ to 1×10¹² cells. In particular embodiments, a composition can have 1×10⁸ HSV-2-reactive CD8 and/or HSV-2-reactive CD4 T cells or more, 1:10⁹ HSV-2-reactive CD8 and/or HSV-2-reactive CD4 T cells or more, 1×10¹⁰ HSV-2-reactive CD8 and/or HSV-2-reactive CD4 T cells or more, 1×10¹¹ HSV-2-reactive CD8 and/or HSV-2-reactive CD4 T cells or more, or 1×10¹² HSV-2-reactive CD8 and/or HSV-2-reactive CD4 T cells or more.

In particular embodiments, the cells are administered to a subject in the form of a pharmaceutical composition, such as a composition including the cells or cell populations and a pharmaceutically acceptable carrier or excipient. The pharmaceutical compositions in some embodiments additionally include pharmaceutically acceptable salts. Suitable pharmaceuticaily acceptable salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

There are a variety of suitable formulations for a pharmaceutical composition including HSV-2-reactive CD8 and/or HSV-2-reactive CD4 T cells. For example, the pharmaceutical composition can contain preservatives. Suitable preservatives may include, for example, methylparaben, propylparaben, sodium benzoate, and benzalkonium chloride. In particular embodiments, a mixture of two or more preservatives is used. The preservative or mixtures thereof are typically present in an amount of 0.0001% to 2% by weight of the total composition.

In addition, buffering agents in particular embodiments are included in the pharmaceutical composition including HSV-2-reactive CD8 and/or HSV-2-reactive CD4 T cells. Suitable buffering agents include, for example, citric acid, sodium citrate, phosphoric acid, potassium phosphate, and various other acids and salts. In particular embodiments, a mixture of two or more buffering agents can be used. The buffering agent or mixtures thereof are typically present in an amount of 0.001% to 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

In particular embodiments, the pharmaceutical composition including HSV-2-reactive CD8 and/or HSV-2-reactive CD4 T cells is formulated as an inclusion complex, such as cyclodextrin inclusion complex, or as a liposome. Liposomes can serve to target the T cells to a particular tissue. Many methods are available for preparing liposomes, such as those described in, for example, Szoka et al., Ann. Rev. Biophys. Bioeng., 9: 467 (1980); U.S. Pat. No. 4,235,871; US 4,501,728; U.S. Pat. 4,837,028; and U.S. Pat. No. 5,019,369.

In particular embodiments, the pharmaceutical composition including HSV-2-reactive CD8 and/or HSV-2-reactive CD4 T cells employs time-released, delayed release, and/or sustained release delivery systems, such that the delivery of the composition occurs prior to, and with sufficient time to cause, sensitization of the site to be treated. Many types of release delivery systems are available and known to those of ordinary skill in the art. Such systems in particular embodiments can avoid repeated administrations of the composition, thereby increasing convenience to the subject and the physician.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

Methods of Use. Methods disclosed herein include treating subjects (e.g., humans, veterinary animals (dogs, cats, birds) livestock (e.g., horses, cattle, goats, pigs) and research animals (e.g., monkeys, rats, mice) with compositions disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. For example, an effective amount can provide an immunogenic effect. Effective amounts are often administered for research purposes. Effective amounts disclosed herein can cause a statistically-significant effect in an in vitro assay, an animal model or clinical study relevant to the assessment of an infection's development, progression, and/or resolution, as well as the effects of the infection. An immunogenic composition can be provided in an effective amount, wherein the effective amount stimulates an immune response.

Conventional techniques can be used to confirm the in vivo efficacy of the identified HSV antigens. For example, one technique makes use of a mouse challenge model. Those skilled in the art, however, will appreciate that these methods are routine, and that other models can be used.

Once a compound or composition to be tested has been prepared, the mouse or other subject is immunized with a series of injections. For example, up to 10 injections can be administered over the course of several months, typically with one to 4 weeks elapsing between doses. Following the last injection of the series, the subject is challenged with a dose of virus established to be a uniformly lethal dose. A control group receives placebo, while the experimental group is actively vaccinated. Alternatively, a study can be designed using sublethal doses. Optionally, a dose-response study can be included. The end points to be measured can include quantitative viral cultures of key organs. The quantity of virus present in tissue samples can be measured. Compositions can also be tested in previously infected animals for reduction in recurrence to confirm efficacy as a therapeutic vaccine.

Efficacy can be determined by calculating the IC50, which indicates the micrograms of vaccine per kilogram body weight required for protection of 50% of subjects from death. The IC50 will depend on the challenge dose employed. In addition, one can calculate the LD50, indicating how many infectious units are required to kill one half of the subjects receiving a particular dose of vaccine. Determination of post mortem viral titer provides confirmation that viral replication was limited by the immune system.

A subsequent stage of testing would be a vaginal inoculation challenge. For acute protection studies, mice can be used. Because they can be studied for both acute protection and reduction or prevention of recurrence, guinea pigs provide a more physiologically relevant subject for extrapolation to humans. In this type of challenge, a non-lethal dose is administered, the guinea pig subjects develop lesions that heal and recur. Measures can include both acute disease amelioration and recurrence of lesions. The intervention with vaccine or other composition can be provided before or after the inoculation, depending on whether one wishes to study reduction or prevention of recurrence versus therapy.

A “prophylactic treatment” includes a treatment administered to a subject who does not display signs or symptoms of an infection or displays only early signs or symptoms of an infection such that treatment is administered for the purpose of diminishing or decreasing the risk of developing the infection further. Thus, a prophylactic treatment functions as a preventative treatment against an infection and/or the potential effects of an infection (e.g., viral reactivation, viral outbreaks) or to reduce infection and/or the potential effects of an infection.

In particular embodiments, a prophylactic treatment can prevent, delay, or reduce the risk of primary infection with a virus. Primary infection can refer to when an HSV-2 seronegative individual first becomes infected by HSV-2 and therefore becomes HSV-2 seropositive.

In particular embodiments, prophylactic treatments reduce, delay, or prevent the worsening of an infection. In particular embodiments, a prophylactic treatment can prevent, delay or reduce the severity of HSV-2 reactivation.

Particular uses of the compositions include use as prophylactic vaccines. Vaccines increase the immunity of a subject against a particular infection. Therefore, “HSV-2 vaccine” can refer to a treatment that increases the immunity of a subject against HSV-2. Therefore, in particular embodiments, a vaccine may be administered prophylactically, for example to a subject that is immunologically naive (e.g., no prior exposure or experience with HSV-2 or currently dormant HSV-2). In particular embodiments, a vaccine may be administered therapeutically to a subject who has been exposed to HSV-2.

In particular embodiments, an HSV-2 vaccine is a therapeutically effective composition including one or more HSV-2 epitopes that elicit or increase the number of TRM that bind the epitopes at sites of viral reactivation. The skilled artisan will appreciate that the immune system generally is capable of producing an innate immune response and an adaptive immune response. An innate immune response generally can be characterized as not being substantially antigen or epitope specific and/or not generating immune memory. An adaptive immune response can be characterized as being substantially antigen specific, maturing over time (e.g., increasing affinity and/or avidity for antigen), and in general can produce immunologic memory. Even though these and other functional distinctions between innate and adaptive immunity can be discerned, the skilled artisan will appreciate that the innate and adaptive immune systems can be integrated and therefore can act in concert.

In particular embodiments, administration of an HSV-2 vaccine can further include administration of one or more adjuvants, for example, if the HSV-2 vaccine is not formulated together with one or more adjuvants or if an additional dose of an adjuvant is deemed beneficial. Exemplary adjuvants are described above.

“Immune response” refers to a response of the immune system to an HSV-2 epitope disclosed herein. In particular embodiments, an immune response to an HSV-2 epitope can be an innate and/or adaptive response. In particular embodiments, an adaptive immune response can be a “primary immune response” which refers to an immune response occurring on the first exposure of a “naive” subject to an HSV-2 epitope.

A “therapeutic treatment” includes a treatment administered to a subject who displays symptoms or signs of an infection and is administered to the subject for the purpose of diminishing or eliminating those signs or symptoms of the infection or effects of the infection (e.g. viral reactivation). The therapeutic treatment can reduce, control, or eliminate the presence or activity of the infection and/or reduce, control or eliminate side effects of the infection.

In particular embodiments a therapeutic treatment can reduce, control, or eliminate HSV-2 reactivation. In particular embodiments, a reduction in HSV-2 reactivation can be determined by measuring expression of HSV-2 latency genes, wherein detection of fewer latency genes or detection of lower expression levels of latency genes can indicate a reduction in HSV-2 reactivation.

In particular embodiments a therapeutic treatment can reduce, control, or eliminate a primary infection with HSV-2.

Function as an effective amount, prophylactic treatment or therapeutic treatment are not mutually exclusive, and in particular embodiments, administered dosages may accomplish more than one treatment type.

In particular embodiments, therapeutically effective amounts provide anti-infection effects. Anti-infection effects include a decrease in the number of infected cells, a decrease in volume of infected tissue, reduced infection-associated and/or reduction or elimination of a symptom associated with the treated infection.

In particular embodiments, therapeutically effective amounts inhibit alphaherpesvirus replication, kill alphaherpesvirus-infected cells, increase secretion of lymphokines having antiviral and/or immunomodulatory activity, and/or enhance production of herpes-specific antibodies.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of infection, stage of infection, effects of infection, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

The pharmaceutical compositions described herein can be administered by, for example, injection, inhalation, infusion, perfusion, lavage or ingestion. Routes of administration can include intravenous, intradermal, intraarterial, intraparenteral, intranasal, intranodal, intralymphatic, intraperitoneal, intralesional, intraprostatic, intravaginal, intrarectal, topical, intrathecal, intratumoral, intramuscular, intravesicular, oral, subcutaneous, and/or sublingual administration and more particularly by intravenous, intradermal, intraarterial, intraparenteral, intranasal, intranodal, intralymphatic, intraperitoneal, intralesional, intraprostatic, intravaginal, intrarectal, topical, intrathecal, intratumoral, intramuscular, intravesicular, oral, subcutaneous, and/or sublingual injection. In particular embodiments, an HSV-2 vaccine can be administered to genital skin or the female reproductive tract to elicit local TRM.

Particular embodiments include contacting an HSV-infected cell with an immune cell directed against an epitope of the disclosure, for example, as described in Tables 1 and 2. Contacting can be performed in vitro or in vivo. In particular embodiments, the immune cell is a T cell. In particular embodiments, the T cells include CD8 and/or HSV-2-reactive CD4 T cells. Compositions of the disclosure can also be used as a tolerizing agent against immunopathologic disease.

Particular embodiments include producing immune cells directed against an alphaherpesvirus, such as HSV. These methods can include contacting an immune cell with an alphaherpesvirus polypeptide of the disclosure. The immune cell can be contacted with the polypeptide via an antigen-presenting cell, wherein the antigen-presenting cell is modified to present an antigen included in a polypeptide of the disclosure. In particular embodiments, the antigen-presenting cell is a dendritic cell. The cell can be modified by, for example, peptide loading or genetic modification with a nucleic acid sequence encoding the polypeptide. In particular embodiments, the immune cell is a T cell. In particular embodiments, the immune cell is a CD8 T cell and/or a CD4 T cell. Also provided are immune cells produced by these methods. The immune cells can be used to inhibit HSV replication, to kill HSV-infected cells, in vitro or in vivo, to increase secretion of lymphokines having antiviral and/or immunomodulatory activity, to enhance production of herpes-specific antibodies, and/or in the treatment, reduction, or prevention of HSV infection in a subject.

In particular embodiments, the pharmaceutical composition includes HSV-2-reactive CD8 and/or HSV-2-reactive CD4 T cells in an amount that is effective to treat, reduce, or prevent HSV-2 infection, such as a therapeutically effective or prophylactically effective amount. Thus, in particular embodiments, the methods of administration include administration of the HSV-2-reactive CD8 and/or HSV-2-reactive CD4 T cells and populations at effective amounts. The appropriate dosage may depend on the severity and course of the infection, whether the HSV-2-reactive CD8 and/or HSV-2-reactive CD4 T cells are administered for prophylactive or therapeutic purposes, previous therapy, the subjects clinical history and response to the cells, and the discretion of the attending physician. Therapeutic or prophylactic efficacy in some embodiments is monitored by periodic assessment of treated subjects. The compositions including HSV-2-reactive CD8 and/or HSV-2-reactive CD4 T cells are in particular embodiments suitably administered to the subject at one time or over a series of treatments. For repeated administrations over several days or longer, the treatment is repeated until a desired suppression of disease symptoms occurs. In particular embodiments, administration can include 1 or 2 rounds of treatment several (e.g., 2-4) weeks apart. However, other dosage regimens may be useful and can be determined.

In particular embodiments, the HSV-2-reactive CD8 and/or HSV-2-reactive CD4 T cells are administered at a desired dosage, which can include a desired dose or number of cells or cell type(s) and/or a desired ratio of cell types. Thus, the dosage of cells in particular embodiments is based on a total number of cells (or number per kg body weight) and a desired ratio of the individual populations or subtypes, such as the CD4 to CD8 ratio. In particular embodiments, the dosage of cells is based on a desired total number (or number per kg of body weight) of cells in the individual populations or of individual cell types. In particular embodiments, the dosage is based on a combination of such features, such as a desired number of total cells, desired ratio, and desired total number of cells in the individual populations.

In particular embodiments, the HSV-2-reactive CD8 and/or HSV-2-reactive CD4 T cells are administered to the subject at a range of one million to 100 billion cells. In particular embodiments, the HSV-2-reactive CD8 and/or CD4 cells are administered to the subject at a range of 1 million to 50 billion cells. In particular embodiments, the HSV-2-reactive CD8 and/or CD4 cells are administered to the subject at a range of 5 million cells, 10 milion cells, 20 million cells, 25 million cells, 30 million cells, 40 million cells, 60 million cells, 70 million cells, 80 million cells, 90 million cells, 100 million cells, 200 million cells, 300 million cells, 400 million cells, 500 million cells, 1 billion cells, 5 billion cells, 10 billion cells, 20 billion cells, 25 billion cells, 30 billion cells, 40 billion cells, 50 billion cells, 75 billion cells, 90 billion cells, 100 billion cells, or more. In particular embodiments, the HSV-2-reactive CD8 and/or CD4 cells are administered to the subject at a range of 100 million cells to 50 billion cells. In particular embodiments, the HSV-2-reactive CD8 and/or CD4 cells are administered to the subject at a range of 120 million cells, 250 million cells, 350 million cells, 450 million cells, 650 million cells, 800 million cells, 900 million cells, 3 billion cells, 30 billion cells, 45 billion cells, or more.

In particular embodiments, the dose of HSV-2-reactive CD8 and/or HSV-2-reactive CD4 T cells is within a range of between 10⁴ and 10⁹ cells/kg body weight, such as between 10⁵ and 10⁶ cells/kg body weight, at 1×10⁵ cells/kg body weight, 1.5×10⁵ cells/kg body weight, 2×10⁵ cells/kg body weight, or 1×10⁶ cells/kg body weight.

In particular embodiments, HSV-2-reactive CD8 and/or HSV-2-reactive CD4 T cells are administered at 1×10⁶ cells, 2.5×10⁶ cells, 5×10⁶ cells, 7.5×10⁶ cells, 9×10⁶ cells, or more. In particular embodiments, HSV-2-reactive CD8 and/or HSV-2-reactive CD4 T cells are administered between 10⁸ and 10¹² cells or between 10¹⁰ and 10¹¹ cells.

In particular embodiments, the cells are administered at a desired output ratio of CD4 and CD8 cells. In particular embodiments, the desired ratio can be a specific ratio or can be a range of ratios. In particular embodiments, the desired ratio of CD4 to CD8 cells can be between 1:5 and 5:1, or between 1:3 and 3:1, or between 2:1 and 1:5. In particular embodiments, the desired ratio of CD4 to CDS cells can be 5:1, 4,5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In particular embodiments, HSV-2-reactive CD8 T cells are administered without HSV-2-reactive CD4 T cells. In particular embodiments, HSV-2-reactive CD4 T cells are administered without HSV-2-reactive CD8 T cells.

HSV-2-reactive CD8 and/or HSV-2-reactive CD4 T cells for adoptive cell transfer can be administered by any suitable means, for example, by bolus infusion, by injection, e.g., intravenous or subcutaneous injections. In particular embodiments, they are administered by parenteral, intrapulmonary, and intranasal, and, if desired for local treatment, intralesional administration. Parenteral infusions include intramuscular, intravenous, intraarterial, intraperitoneal, intrathoracic, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In particular embodiments, HSV-2-reactive CD8 and/or HSV-2-reactive CD4 T cells are administered as part of a combination treatment, such as simultaneously with or sequentially with, in any order, another therapeutic intervention, such as an antibody or engineered cell or receptor or other agent, such as an anti-viral or therapeutic agent. Thus, the cells in particular embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In particular embodiments, the cells are administered prior to the one or more additional therapeutic agents. In particular embodiments, the cells are administered after the one or more additional therapeutic agents. In particular embodiments, the one or more additional agents includes a cytokine, such as IL-2 or other cytokine, for example, to enhance persistence.

Kits. Also disclosed herein are kits including one or more containers including one or more of HSV-2 vaccine epitopes, HSV-2 therapeutics, HSV-2-reactive CD8 T cells, HSV-2-reactive CD4 T cells, and/or compositions and/or adjuvants described herein. Associated with such container(s) can be a notice in the form prescribed by a governmental agency regulating the manufacture, use, or sale of pharmaceuticals or biological products, which notice reflects approval by the agency of manufacture, use, or sale for human administration. Particular embodiments of kits include: one or more HSV-2 vaccine epitopes; one or more HSV-2 epitopes recognized by CD8 TRM; one or more HSV-2 epitopes recognized by CD4 TRM; one or more HSV-2 epitopes recognized by CD8 TRM and one or more HSV-2 epitopes recognized by CD4 TRM; one or more HSV-2 vaccine epitopes of SEQ ID NOs: 1-22, and 30-34; HSV-2-reactive CD8 T cells; HSV-2-reactive CD4 T cells; HSV-2-reactive CD8 T cells and HSV-2-reactive CD4 T cells. In particular embodiments, kits including an HSV-2 vaccine epitope further includes one or more vaccine adjuvants. In particular embodiments, the adjuvants can include alum, a squalene-based adjuvant, a STING agonist, a liposome-based adjuvant, a saponin-based adjuvant, a stable emulsion of TLR4 agonist glucopyranosyl lipid A (GLA SE), and/or a carbomer-lecithin-based adjuvant. The kits can include a plurality of containers for storing and/or administering HSV-2-reactive CD8 T cells and/or HSV-2-reactive CD4 T cells. For example, a container can include a single unit dose of the cells. The unit dose may be an amount or number of the cells to be administered to the subject in the first dose or twice the number (or more) of the cells to be administered in the first or subsequent dose(s). Exemplary containers include infusion bags, intravenous solution bags, vials, including those with stoppers pierceable by a needle for injection. Kits can further include one or more additional containers with a composition contained therein which includes a further agent, such as an anti-viral or otherwise therapeutic agent, for example, which is to be administered in combination, e.g., simultaneously or sequentially in any order, with the HSV-2 therapeutics or HSV-2-reactive T cells. Kits may further include a pharmaceutically acceptable buffer. Kits may further include other materials such as other buffers, diluents, filters, tubing, needles, and/or syringes.

Particular embodiments include diagnostic assays. The diagnostic assays can be used to identify the immunological responsiveness of a subject suspected of having a herpetic infection and to predict responsiveness of the subject to a particular course of therapy. In particular embodiments, the assays include exposing T cells of a subject to an antigen of the disclosure, in the context of an appropriate APC, and testing for immunoreactivity by, for example, measuring IFNv, proliferation or cytotoxicity. Suitable assays are known in the art.

### Exemplary Embodiments.

1. An HSV-2 vaccine epitope including one or more immunogenic proteins selected from SEQ

ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 30; SEQ ID NO: 31; SEQ ID NO: 32; SEQ ID NO: 33; or SEQ ID NO: 34.
2. A fusion protein including an HSV-2 vaccine epitope of embodiment 1.
3. A fusion protein of embodiment 2 including at least 2 HSV-2 vaccine epitopes of embodiment 1; at least 3 HSV-2 vaccine epitopes of embodiment 1; at least 4 HSV-2 vaccine epitopes of embodiment 1 or at least at least 5 HSV-2 vaccine epitopes of embodiment 1.
4. A fusion protein of embodiment 2 or 3 including a CD8 TRM epitope and a CD4 TRM epitope.
5. A fusion protein of any of embodiments 2-4 including at least two CD8 TRM epitopes and a CD4 TRM epitope.
6. A fusion protein of any of embodiments 2-5 including a CD8 TRM epitope and at least two CD4 TRM epitopes.
7. A fusion protein of any of embodiments 2-6 including a multimerization domain.
8. A fusion protein of embodiment 7 wherein the multimerization domain is a C4b domain.
9. A fusion protein of embodiment 7 wherein the multimerization domain is selected from SEQ ID NO: 23; SEQ ID NO: 24; SEQ ID NO: 25; or SEQ ID NO: 26.
10. A composition including an HSV-2 vaccine epitope or a fusion protein of any of embodiments 1-9.
11. A composition of embodiment 10 wherein the composition is an immunogenic composition.
12. A composition of embodiment 10 or 11 wherein the composition is a therapeutic composition.
13. A composition of any of embodiments 10-12 further including one or more adjuvants.
14. A composition of embodiment 13 wherein the one or more adjuvants are selected from alum, a squalene-based adjuvant, a STING agonist, a liposome-based adjuvant, a saponin-based adjuvant, a stable emulsion of TLR4 agonist glucopyranosyl lipid A (GLA SE), or a carbomer-lecithin-based adjuvant.
15. A method of stimulating an anti-HSV-2 immune response in a subject including administering to the subject a therapeutically effective amount of a composition of any of embodiments 10-14 thereby stimulating an HSV-2 immune response in the subject.
16. A method of embodiment 15 wherein the subject is HSV-2 seropositive.
17. A method of embodiment 15 wherein the subject is an HSV-2 seronegative subject, and wherein the therapeutically effective amount reduces the likelihood of the HSV-2 seronegative subject becoming HSV-2 seropositive.
18. A method of treating a herpes simplex virus type 2 (HSV-2) infection in a subject including administering an HSV-2 vaccine epitope, a fusion protein, or a composition of any of the preceding embodiments to the subject thereby treating the HSV-2 infection in the subject.
19. A method of eliciting CD8 and/or CD4 TRM including administering an HSV-2 vaccine epitope, a fusion protein, or a composition of any of the preceding embodiments.
20. A method of enhancing proliferation of HSV-specific T cells including contacting the HSV-specific T cells with an HSV-2 vaccine epitope, a fusion protein, or a composition of any of the preceding embodiments.
21. A method of inducing an immune response to an HSV infection in a subject including administering an HSV-2 vaccine epitope, a fusion protein, or a composition of any of the preceding embodiments to the subject thereby inducing an immune response to the HSV infection in the subject.
22. A method of treating a herpes simplex virus type 2 (HSV-2) infection in a subject including administering a therapeutically effective amount of HSV-2-reactive CD8 and/or HSV-2-reactive CD4 T cells, or a composition of the cells, to the subject, thereby treating the HSV-2 infection in the subject.
23. A method of embodiment 22, wherein the cells are administered intravenously or subcutaneously.
24. A method of embodiment 22, wherein the cells are reactive to HSV-2 epitopes of SEQ ID NOs: 1-22, and/or 30-34.

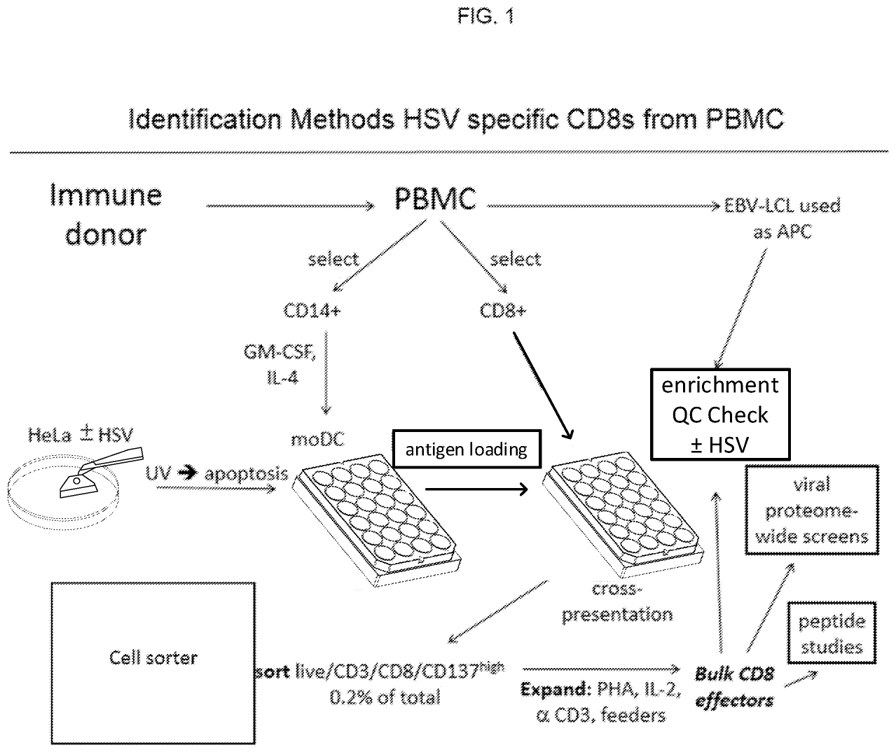

Experimental Methods. Human specimens (see FIG. 1). Written informed consent was obtained from all subjects. Subjects were HIV-uninfected, HSV-2 seropositive, and had a history of genital herpes. Venous blood was processed for PBMC by Ficoll centrifugation and PBMC were cryopreserved. Biopsies of the cervix were obtained using forceps and placed into cell culture media. Skin biopsies of genital skin at sites of previous HSV-2 infection were performed as described in Koelle, et al., Journal of Infectious Diseases 169, 956-961, (1994). The biopsies were digested with collagenase to isolate single cells as described (Posavad et al, Mucosal Immunology 10, 1259-1269 (2017)). HLA typing was performed at Bloodworks Northwest, Seattle, Wash., or by Scisco Genetics, Seattle, Wash.

Cell culture. To create dendritic cells (DC) for use as antigen presenting cells (APC), PBMC were cultured as previously described in Jing, et al., Journal of Clinical Investigation 122, 654-673, (2012). Briefly, adherent cells were cultured in the presence of GM-CSF and IL-4 for 5 to 7 days. HeLa cells were cultured also as described in Jing, et al., Journal of Clinical Investigation 122, 654-673, (2012). Vero cells were cultured in DMEM with 10% fetal calf serum (FSC). EBV-transformed B cell lines (EBV-LCL) were cultured as described in Tigges, et al., Journal of Virology 66, 1622-1634, (1992). Cos7 cells were cultured as described in Koelle, et al., Journal of immunology 166, 4049-4058, (2001). All cells were Mycoplasma negative.

HSV-2 reagents. Stocks of HSV-2 strain 186 (Nishiyama & Rapp, The Journal of General Virology 52, 113-119, (1981)) and HSV-2 strain with deleted gene UL41 were created in Vero cells and titered in Vero cells as described in Koelle, et al., Journal of Infectious Diseases 169, 956-961, (1994). The genome-covering HSV-2 clone collection made from strain 186 DNA in custom vector pDEST103 has been described in Johnston, et al., Journal of Virology 88, 4921-4931, (2014). HSV-2 peptides from the strain 186 predicted proteome were obtained from Genscript and routinely dissolved at 10 mg/ml in DMSO.

Enrichment and expansion of HSV-2-specific T-cells from cervical and skin biopsies. DC were loaded with HeLa cell-derived HSV-2 antigen or mock antigen as previously described for HSV-1 in Jing, et al., Journal of Clinical Investigation 122, 654-673, (2012). The DC preparation was labeled with CFSE as previously described in Jing, et al., Journal of Clinical Investigation 122, 654-673, (2012). The digested suspension of cells from the cervical or skin biopsy was admixed with the DC prep and incubated for 18 hours at 37° C., 5% CO₂, in T-cell medium (TCM) (Jing, et al., Journal of Clinical Investigation 122, 654-673, (2012)). At this time, the combined cells were harvested by centrifugation and stained with mAb specific for human CD3, CD4, CD8, and CD137, and a live/dead viability stain. The cells were analyzed on a FacsAria II cell sorter. From the wells in which HSV-2-loaded DC were used as stimulator cells, putative HSV-2-reactive cells gated sequentially as lymphocyte forward/side scatter, live, responder (CFSE-negative, CD3+, and either CD4+CD8− (CD4 T cells) or CD4-CD8+ (CD8 T cells), that also showed signs of recent activation as detected by high cell surface expression of CD137 (CD4 CD137^{high} T cells and CD8 CD137^{high} T cells) were sterilely collected. As a control group, similar CD4 and CD8 T cells that were CD137 negative were collected. The cells were expanded initially with PHA as mitogen, human natural IL-2 as growth factor, and feeder cells as described in Tigges, et al., Journal of Virology 66, 1622-1634, (1992). For further expansion, the cells were re-expanded with anti-CD3 mAb as mitogen, recombinant IL-2 as growth factor, and feeder cells as described in Koelle, et al., Journal of Immunology 166, 4049-4058, (2001).

T cell functional assays. Quality control checks on the sorted T cell subpopulations were initially performed after the initial round of expansion. These were performed as published in Jing, et al., Journal of Clinical Investigation 122, 654-673, (2012). In brief, for CD8 T cells, autologous EBV-LCL were infected with HSV-2 strain 186 at multiplicity of infection (MOI) 10 for 18 hours or mock infected. CD8 T cells derived from the biopsy and EBV-LCL were co-cultured for 18 hours in the presence of Brefeldin A and co-stimulatory mAbs and then processed for intracellular cytokine secretion (ICS) for accumulation of interferon-gamma (IFN-γ) and IL-2, with analysis by flow cytometry. The EBV-LCL were CFSE labeled to allow dump-gating. For CD4 T cells, the workflow published in Jing, et al., Journal of Clinical Investigation 122, 654-673, (2012) was also used. In brief, CD4 T cells derived from the biopsy and autologous CFSE-labeled PBMC were co-cultured for 18 hours in the presence of UV-killed HSV-2 strain 186 cell-associated viral antigen or mock antigen. Again, Brefeldin A and co-stimulatory mAbs were used and cells processed for ICS. For both CD4 and CD8 biopsy-derived T cells, analysis on a LSRII flow cytometer gated on live, responder, CD3+, and either CD4+or CD8+ T-cells as appropriate, and analyzed the percent of cells staining positive for IFN-γ and/or IL-2 as an indication of specificity for HSV-2.

To determine which HSV-2 open reading frame the biopsy-derived T cells recognized, genome-wide functional screens were performed. The technology has been reported in depth for CD8 T cells for HSV-1 for example, in Jing, et al., Journal of Clinical Investigation 122, 654-673, (2012). A similar workflow was used for CD8 T cells to query the HSV-2 genome, except that a collection of every known HSV-2 gene was used. The gene set has been previously reported in Johnston, et al., Journal of Virology 88, 4921-4931, (2014). Each HSV-2 gene was subcloned into pDEST103 for CD8 research (Jing, et al., Journal of Clinical Investigation 122, 654-673, (2012)). For each subject, the HLA class I cDNA molecule for both of their HLA A locus alleles and both of their HLA B locus alleles were cloned. Then, in 96-well plates, Cos-7 cells were co-transfected with subject-specific HLA cDNA and each individual HSV-2 gene. Assays were done in duplicate. After 48 hours, 10⁵ bulk CD8 T cells were added per well. After an additional 24-48 hours, supernatants were harvested and tested by IFN-γ ELISA as published in Koelle, et al., Journal of Immunology 166, 4049-4058, (2001). The presence of IFN-γ above background levels was considered to indicate the presence of CD8 T cells reactive with the relevant HSV-2 gene. Once these initial hits were obtained at the whole HSV-2 gene level, peptides from within that specific gene were tested as described in Jing, et al., Journal of Clinical Investigation 122, 654-673, (2012) at a concentration of 1 μg/ml. These synthetic peptides were picked based on matches to predicted HLA binding motifs (Kim et al. (2011) Journal of immunological methods 374(1-2), 62-69) or in some instances, gene-covering sets of overlapping peptides covering the entire protein sequence were already available and were tested. In each case reported herein, single active HSV-2 peptides were detected. In some instances, dose-response assays were conducted with serial dilutions of peptide to find the 50% effective concentrations, as previously described in Jing, et al., Journal of Clinical Investigation 122, 654-673, (2012).

The technology used to determine the fine peptide specificity of biopsy-derived CD4 T cells that recognize HSV-2 has been described in Johnston, et al., Journal of Virology 88, 4921-4931 (2014). In brief, every HSV-2 was expressed. The bulk biopsy-derived CD4 T cells were admixed with autologous PBMC and each protein in duplicate. T cell activation was detected by proliferation or IFN-γ secretion assays. Once the identity of reactive HSV-2 proteins were determined, peptides from within that protein were tested in repeat assays as described in Johnston, et al., Journal of Virology 88, 4921-4931, (2014). In some cases, dose-response assays were conducted as described in Jing, et al., Journal of Immunology 196, 2205-2218, (2016).

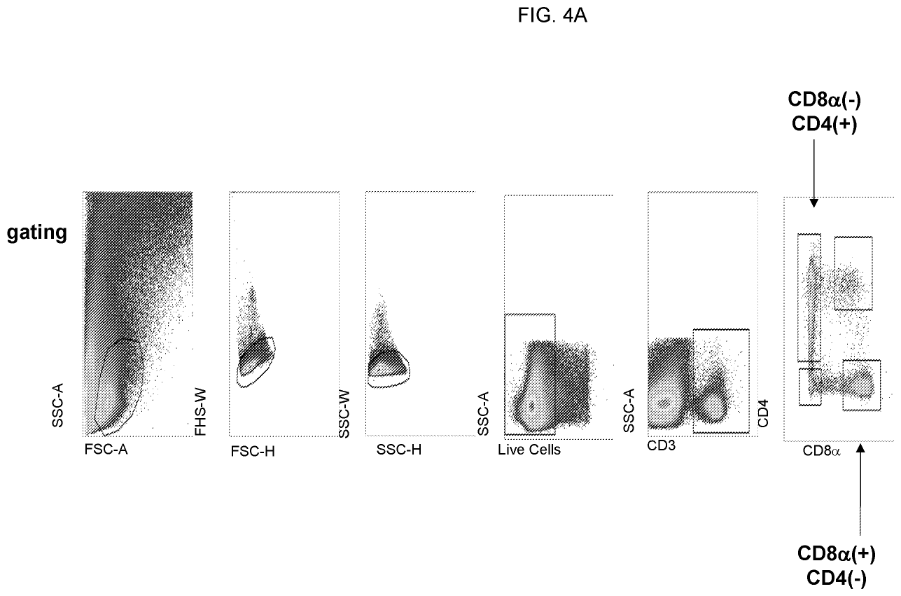

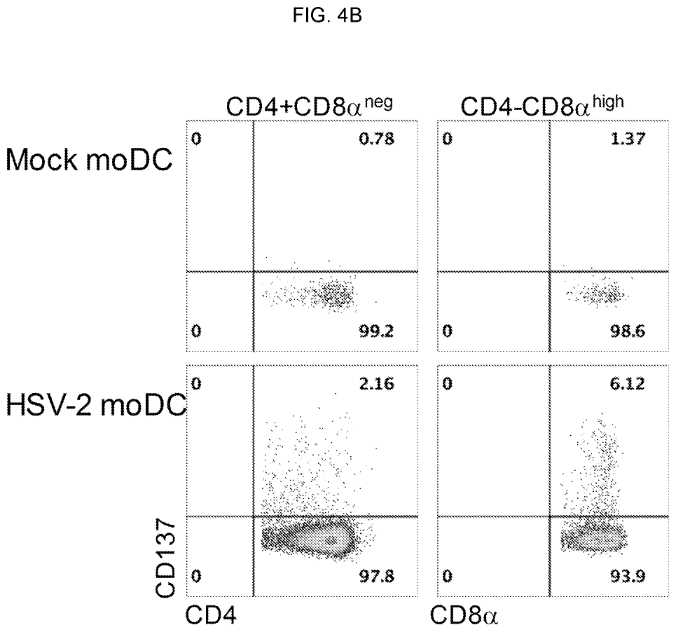

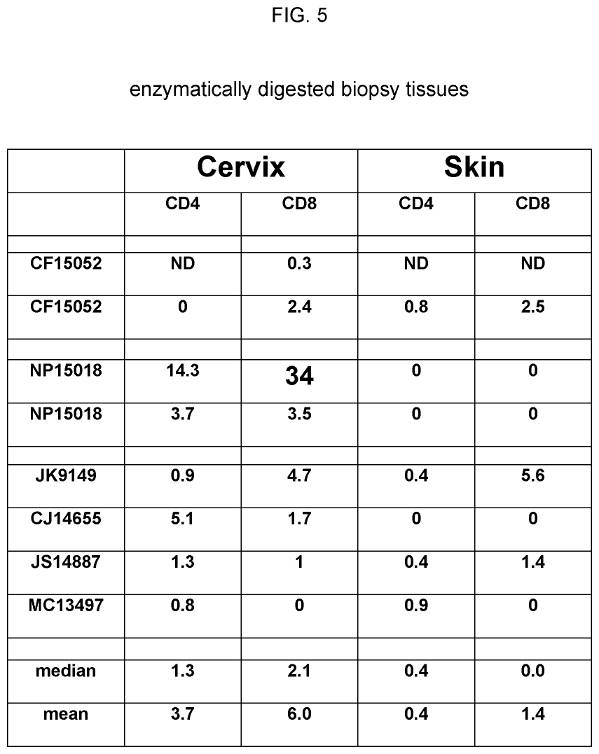

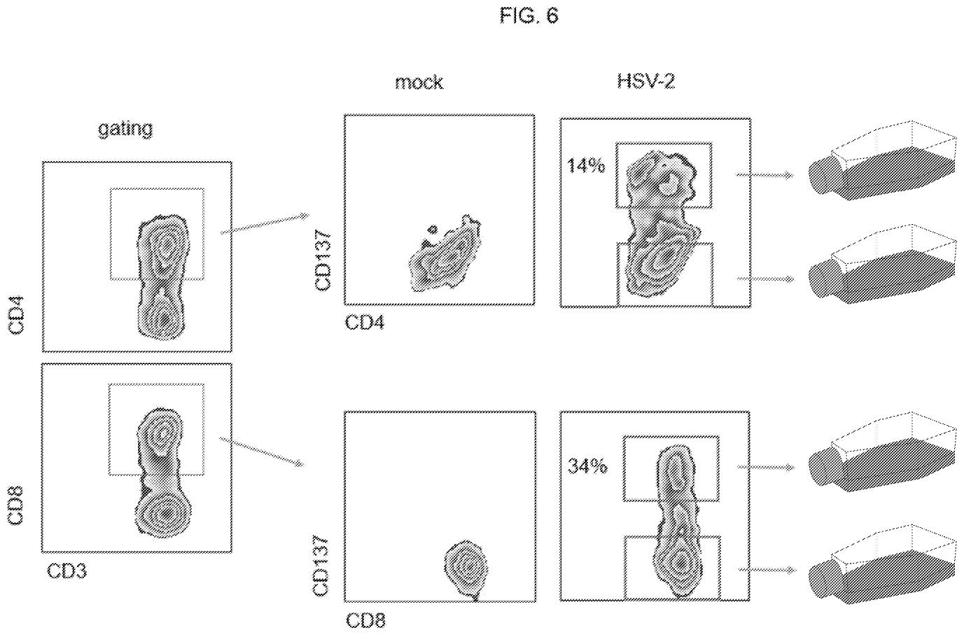

Experimental Results. Putative HSV-2 reactive T cells were identified as described in Jing et al. (2012) and in FIG. 1 above. FIG. 4B shows results of expression of CD137, an activation marker expressed on the surface of cervical biopsy T cells after they have been activated through their T cell receptor, when the biopsy cells are co-incubated with DC that were either treated with mock virus (top) or HSV-2 (bottom) in the form of infected HeLA cells prepared for cross-presentation as outlined in Jing et al. (2012). The left column shows biopsy CD4 T cells and the right column shows biopsy CD8 T cells. As can be seen, the biopsy CD4 T cells and biopsy CD8 T cells (the left-hand and right-hand boxes, respectively) co-incubated with DC treated with HSV-2-infected HeLA cells (bottom row) have increased surface CD137 expression, as shown by the increased percentage of CD137+ cells, when compared to biopsy CD4 T cells and biopsy CD8 T cells (the left-hand and right-hand boxes, respectively) co-incubated with DC that have been mock-treated (top row), consistent with the presence of HSV-specific TRM in the biopsy cell preparation. The table in FIG. 5 summarizes HSV-2-reactive data for biopsy-derived CD4 or CD8 T cells from the indicated subjects and shows that TRM directed net ex vivo reactivity to whole HSV-2. FIG. 6 shows abundant HSV-2-reactive CD4 and CD8 T-cells from subject NP15018 in FIG. 5. A much higher proportion of the T cells express CD137 when they were exposed to DC treated with HSV-2-infected HeLa cells. The sorted CD137-high cells were physically separated from the CD4 T cells and from the CD8 T cells using a cell sorter for expansion.

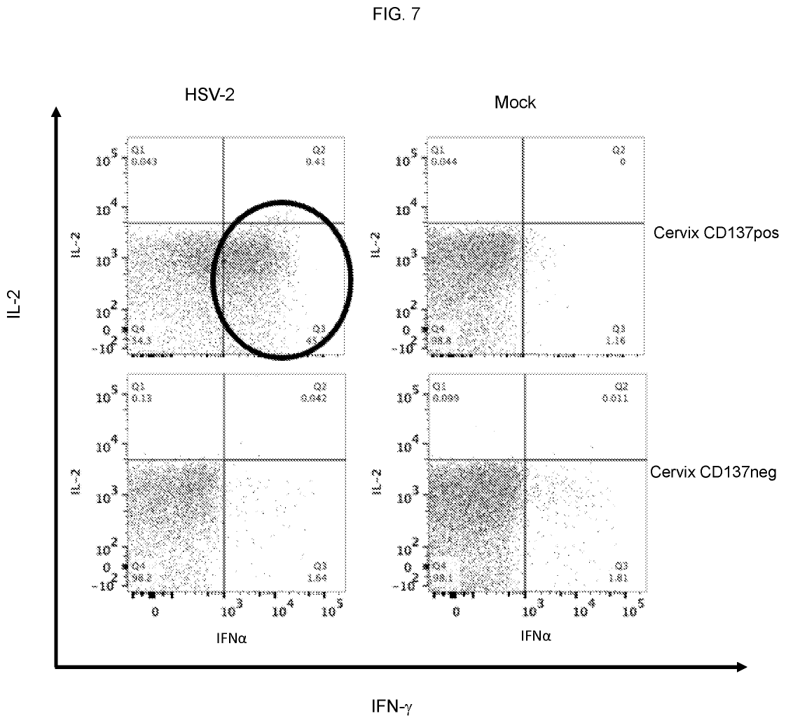

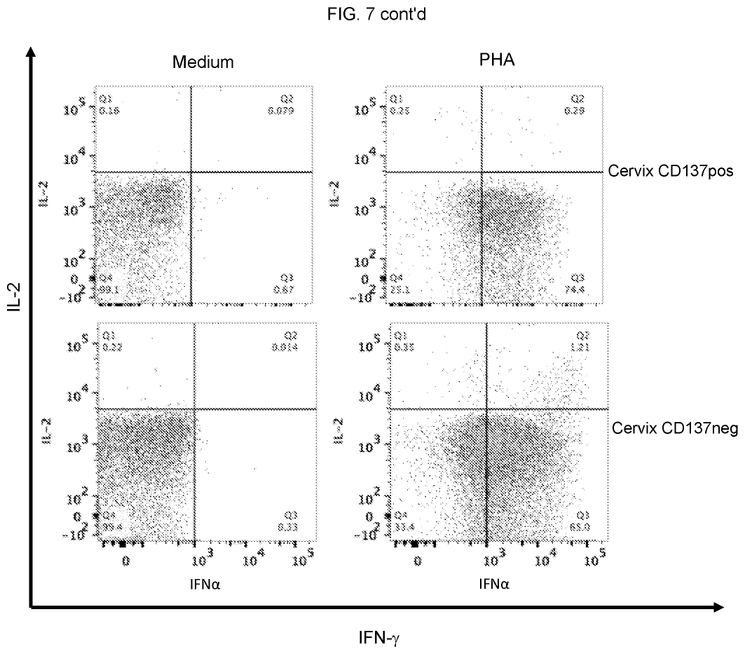

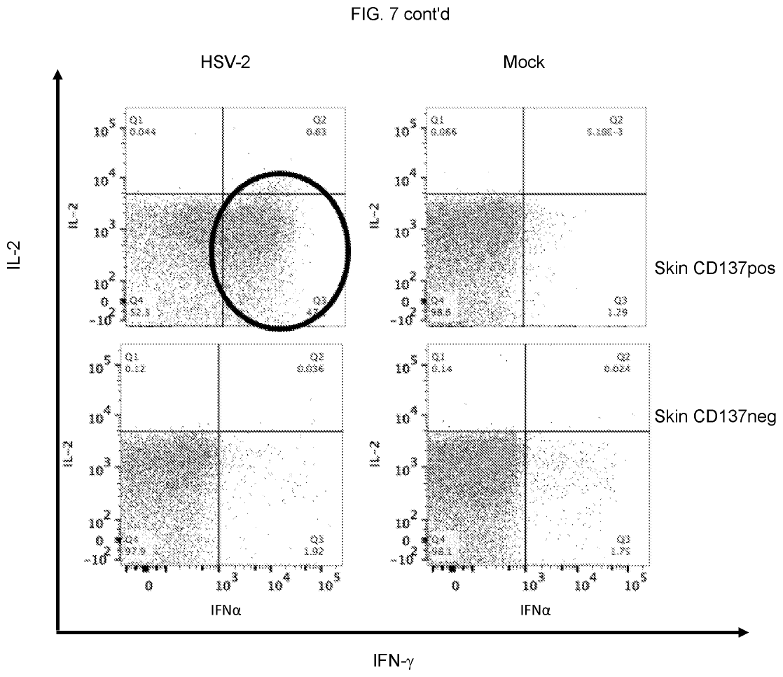

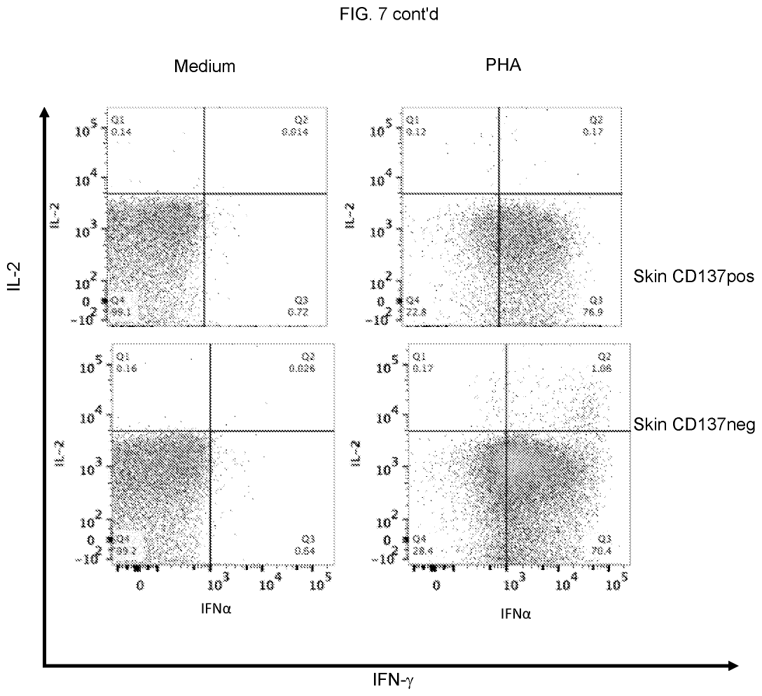

The response of cervix or skin CD137-high CD8 T cells to HSV-2 was investigated. Briefly, HSV-2 was presented as a test article by infecting autologous, self, patient-matched B lymphocytes with HSV-2 and co-culturing them with the following expanded T cell populations: cervical CD137-positive, cervical CD137-negative, skin CD137-positive, and skin CD137-negative. The activation of these CD8 cells was measured by intracellular cytokine cytometry by measuring the level of IFN-γ (x axis, FIG. 7) and IL-2 (Y axis, FIG. 7) inside the permeabilized, gated cervix or skin T cells (gating scheme not shown). FIG. 7 shows that high activation of cervical or skin CD8 T cells is seen only in the cell cultures that started out as sorted CD137-high cervix or CD137-high skin CD8 T cells. However, all cultures respond to positive control PHA and are non-responsive to the negative controls (‘Mock’ and ‘Medium’).

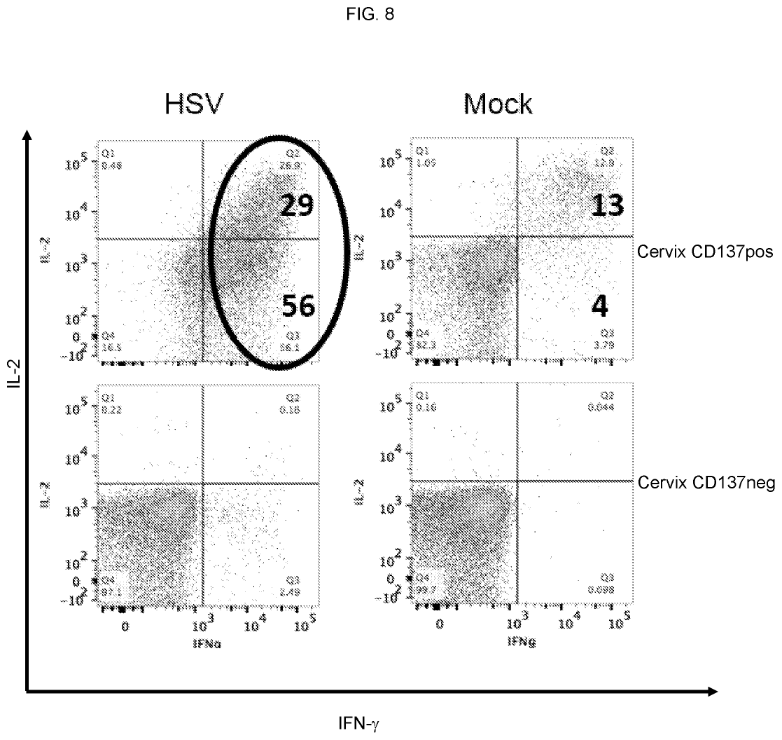

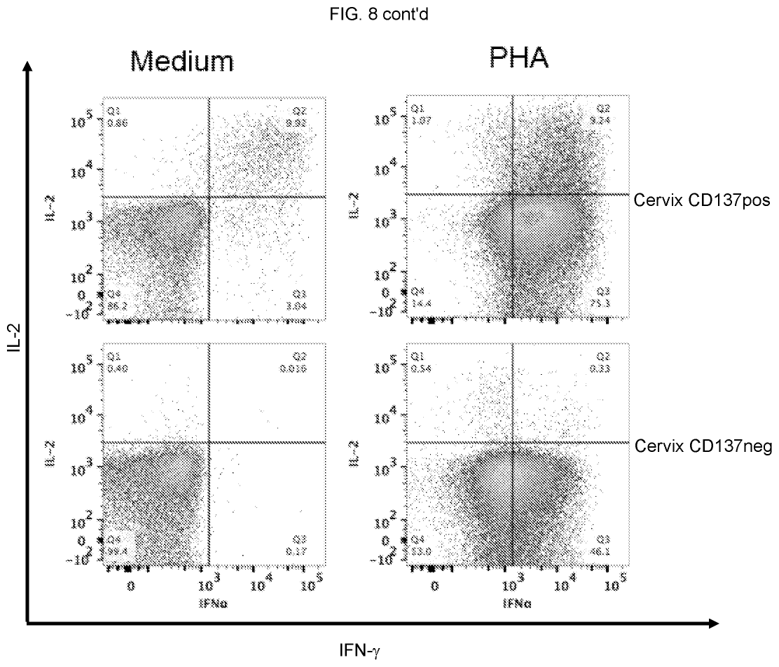

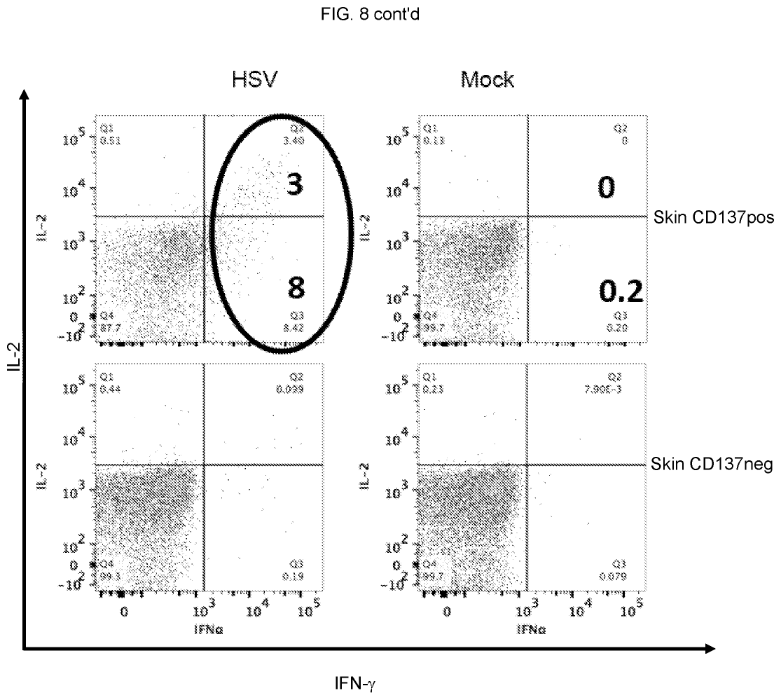

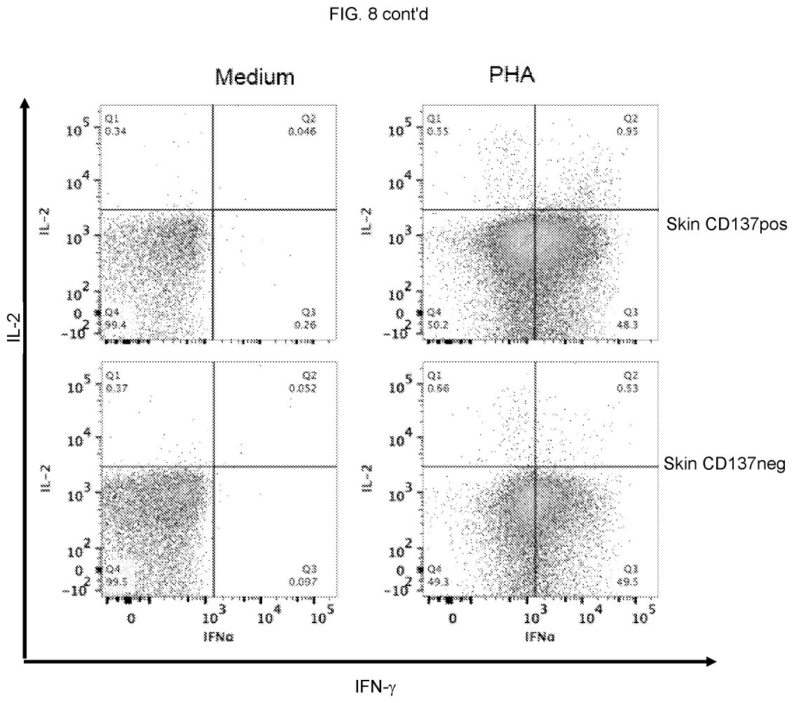

The response of cervix or skin CD137-high CD4 T cells to HSV-2 was investigated following a procedure similar to the one described above for CD137-high CD8 T cells except that whole HSV-2 was added as killed virus, with self, autologous PBMC added as antigen presenting cells (APC). Similar to what was observed with the cervix or skin CD137-high CD8 T cells in FIG. 7, high activation of cervical or skin CD4 T cells is seen only in the cell cultures that started out as sorted CD137-high cervix or CD137-high skin CD4 T cells (FIG. 8).

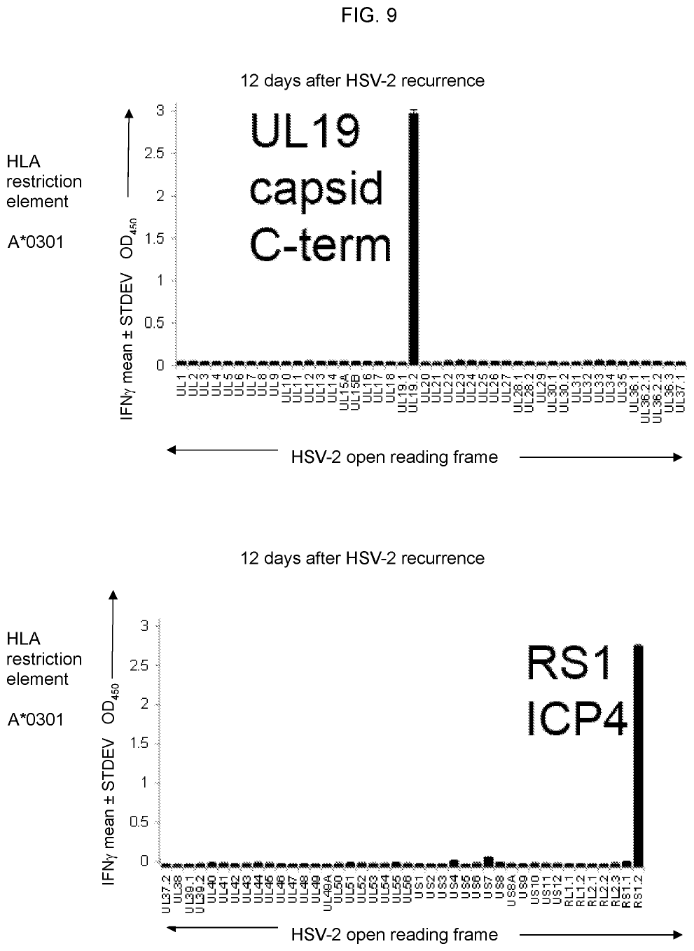

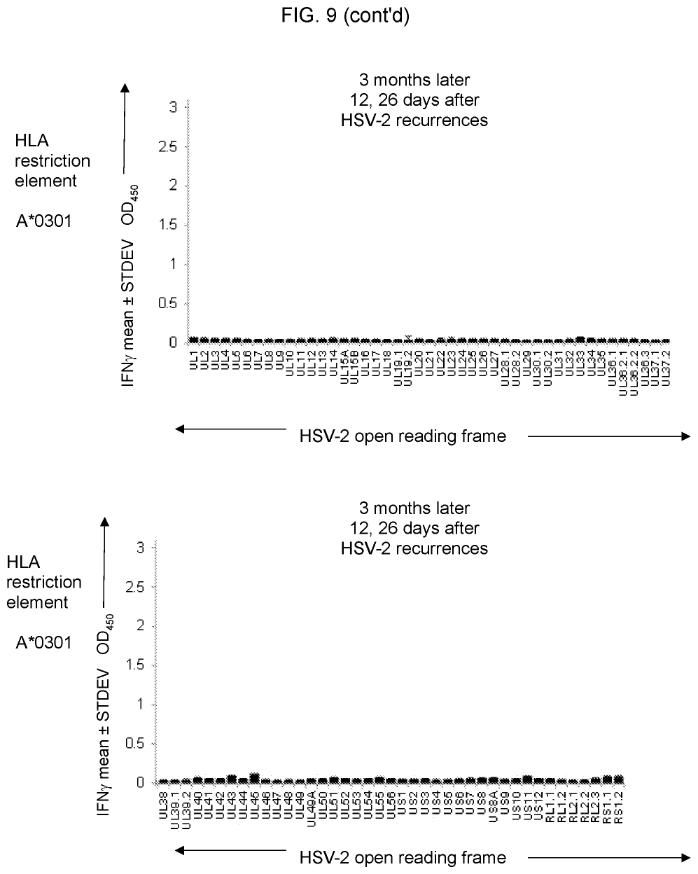

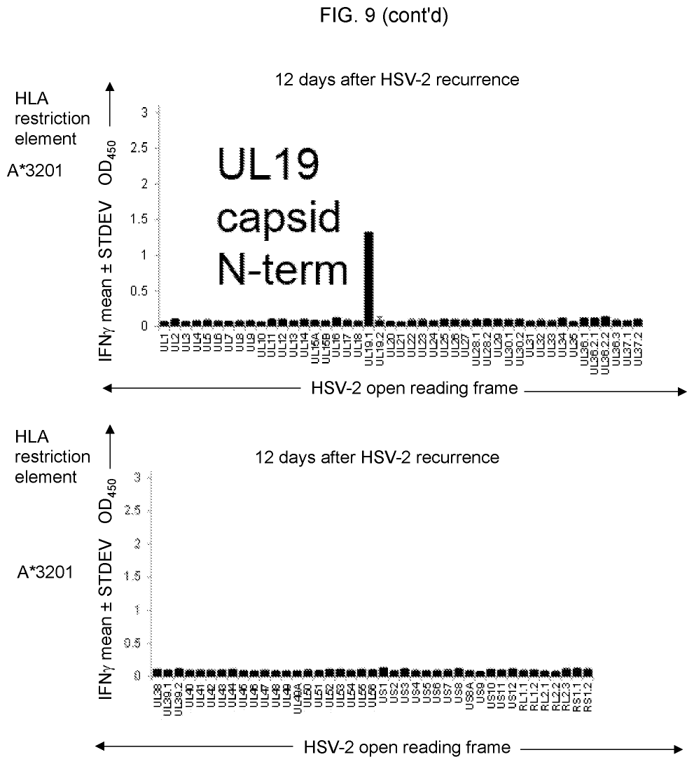

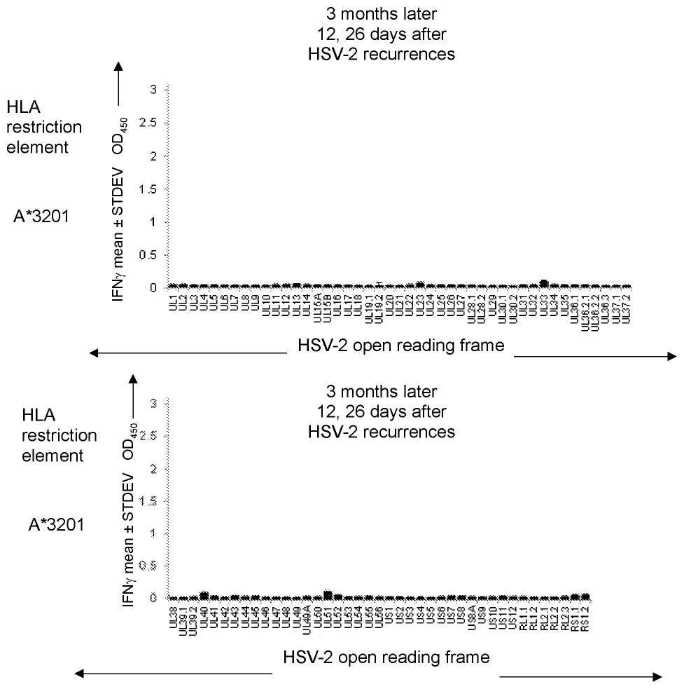

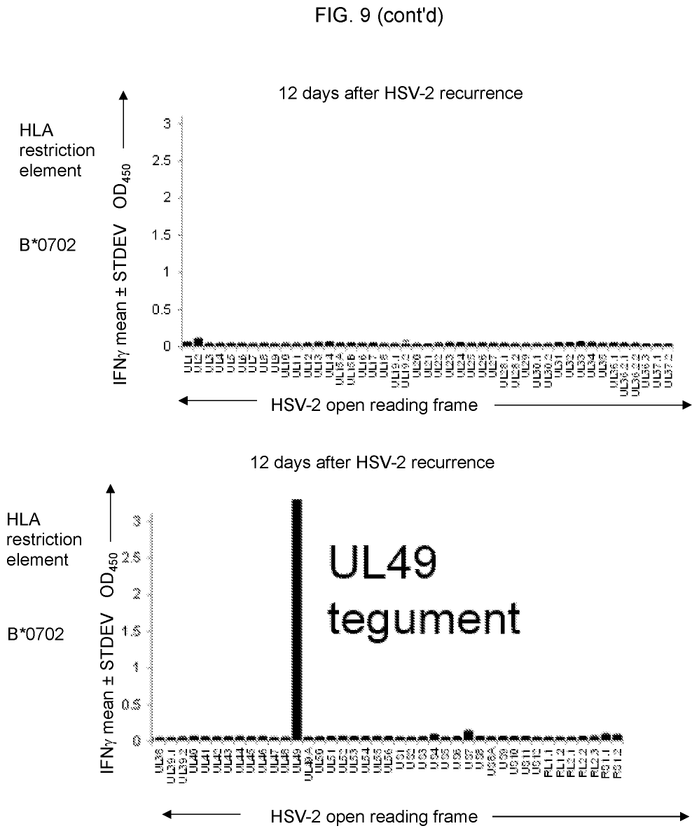

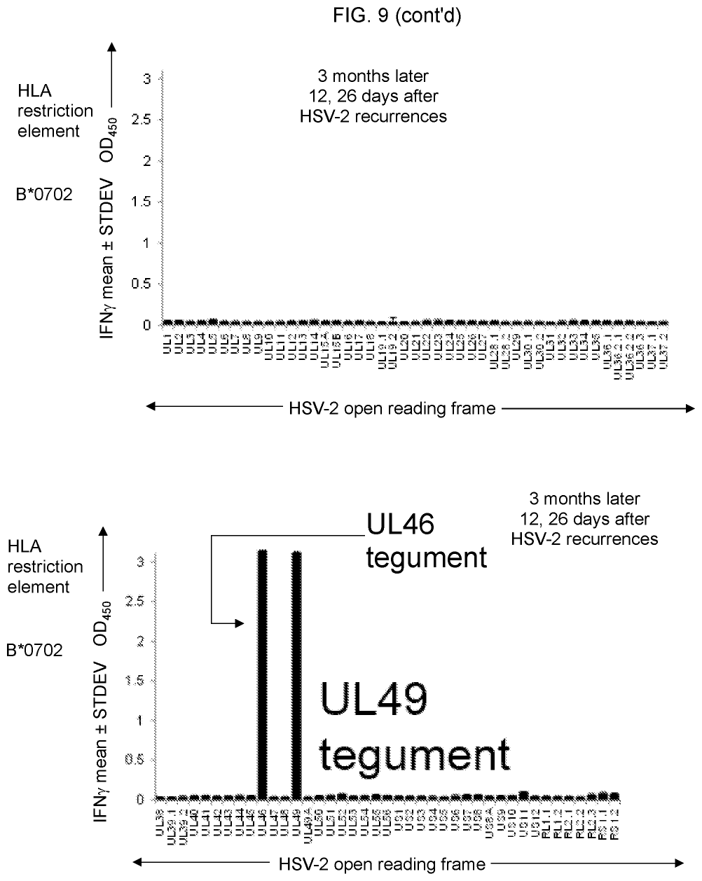

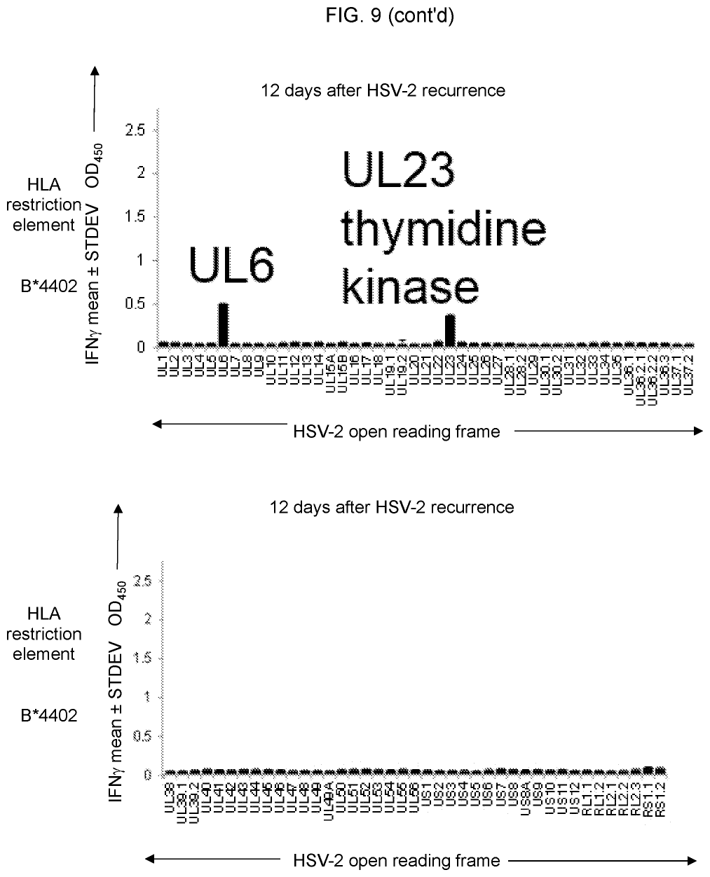

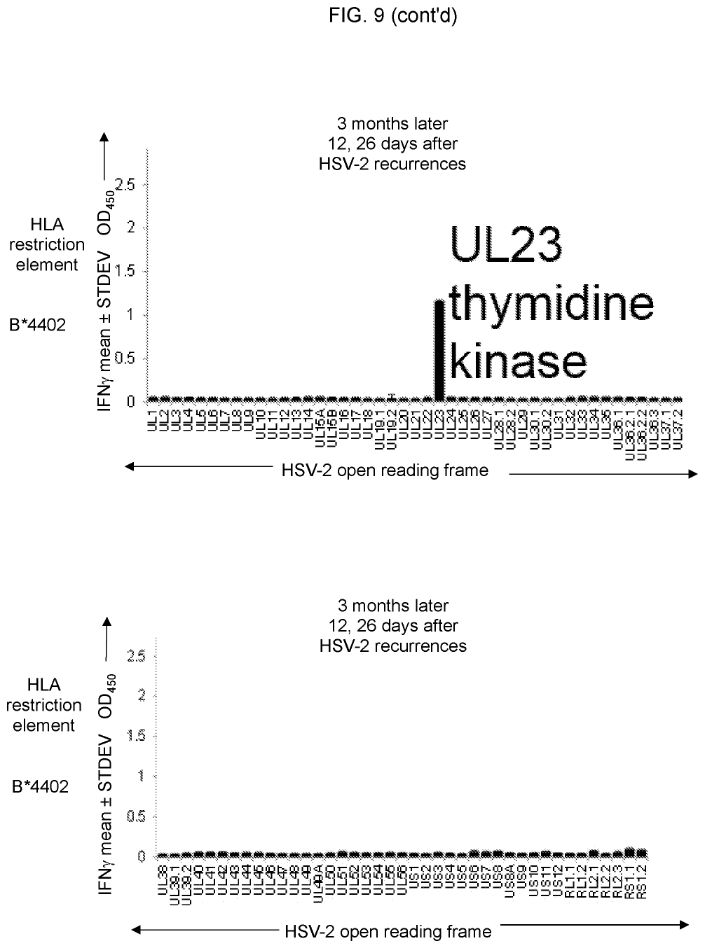

HSV-2 proteome-wide CD8 T_{RM} screens for HLA A, B were performed as described in Jing et al. (2016) and in the description of FIG. 2. FIG. 9 shows reactive HSV-2 genes or gene fragments identified from these screens.

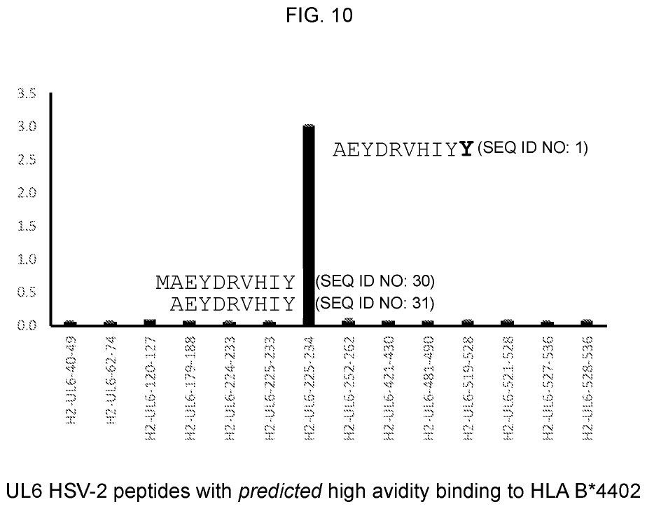

UL6 HSV-2 peptides were made based on a predictive algorithm for binding to HLA B*4402 and tested for their ability to activate CD137 high biopsy-derived CD8 T cells that had been activated in response to HLA B*4402 and HSV-2 gene UL6 in a T cell activation assay described in FIG. 9. FIG. 10 shows UL6 HSV-2 peptides with predicted high avidity binding to HLA B*4402 (AEYDRVHIYY, SEQ ID NO: 1, MAEYDRVHIY, SEQ ID NO: 30, and AEYDRVHIY, SEQ ID NO: 31).

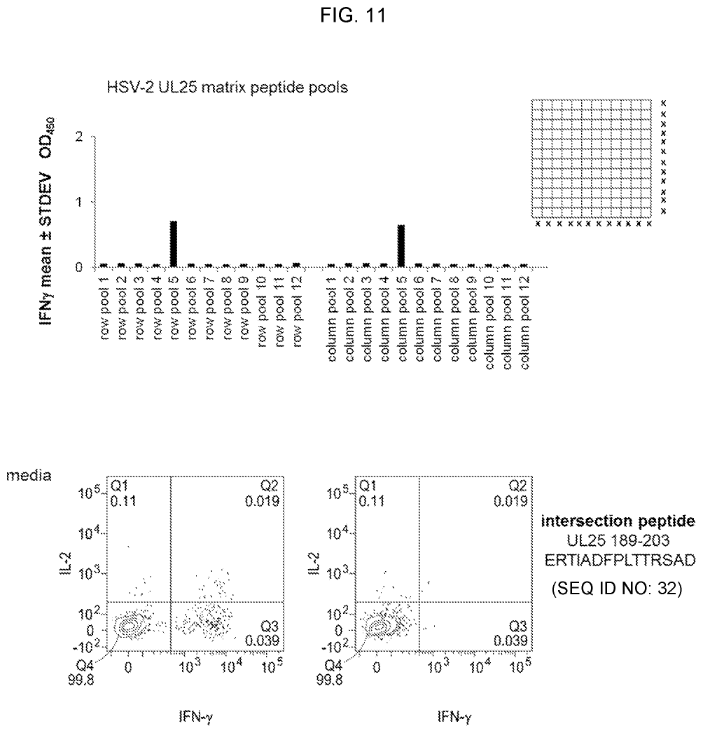

Biopsy-derived CD8 T cells recognizing Cos7 co-transfected with the combination of a subject HLA molecule and HSV-2 UL25 gene were identified. A matrix of UL25 peptide (15 mers, overlapping by 11 AA) pools was created and tested for their ability to activate the biopsy-derived cells. One row and one column pool were positive (FIG. 11). The single peptide at the intersection of the row and column pools (‘UL25 189-203’, ERTIADFPLTTRSAD, SEQ ID NO: 32) elicited an IFN-γ response from the biopsy cells (bottom, right-hand graph of FIG. 11) while the negative media control did not (bottom, left-hand graph of FIG. 11).

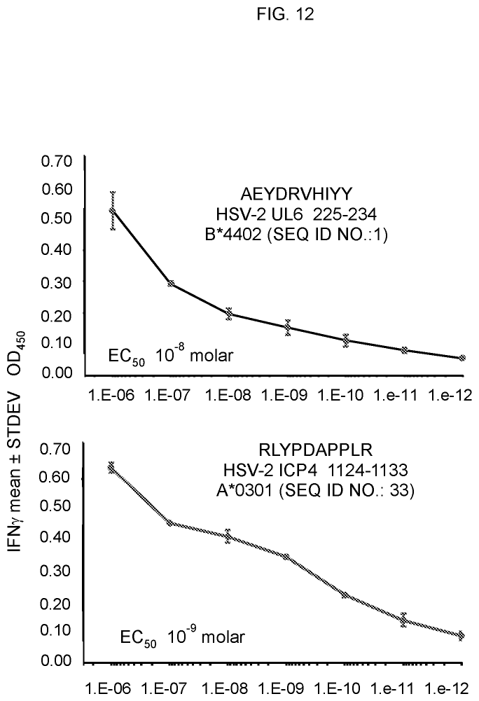

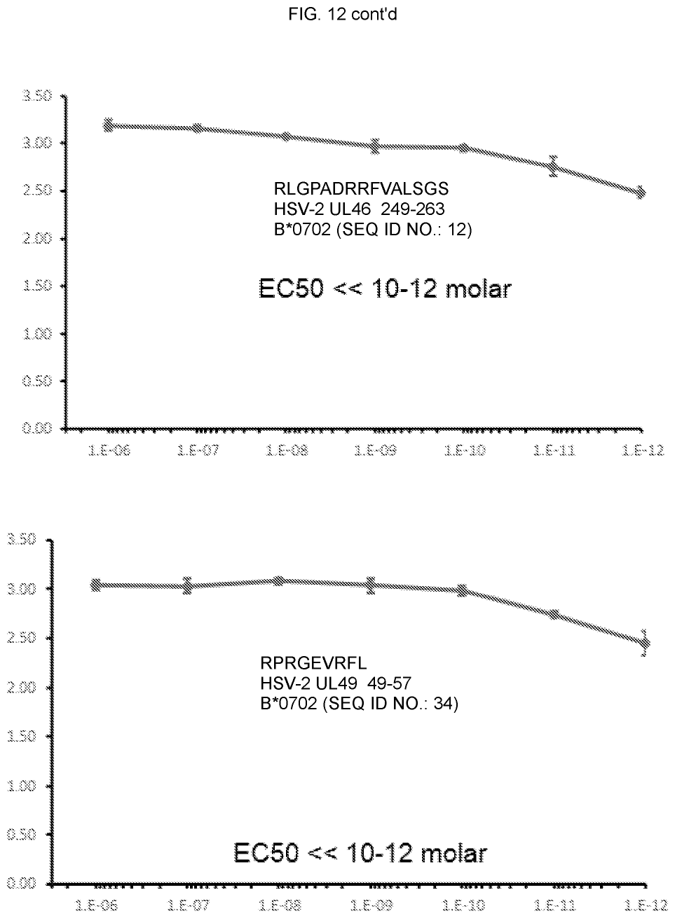

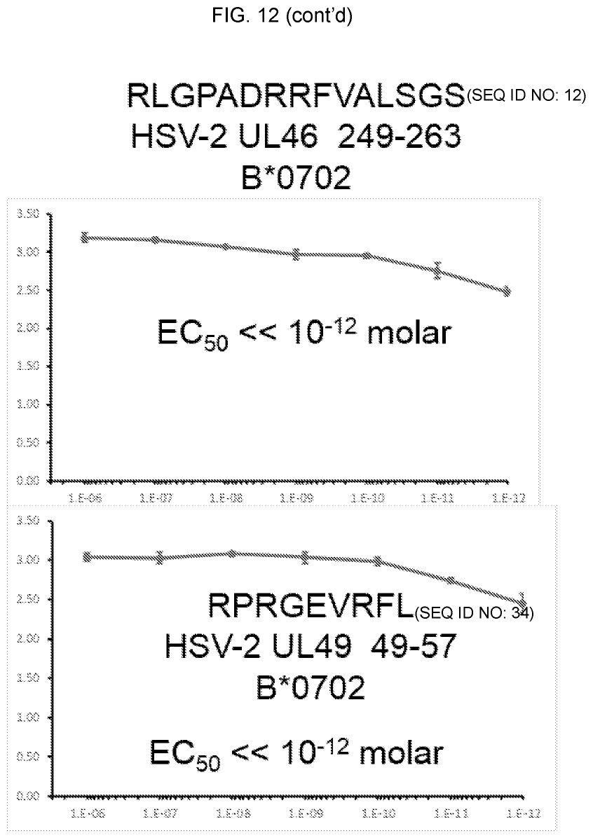

The EC₅₀ value, or concentration of peptide required for 50% triggering of the T cells, was determined for four peptides of the indicated HSV-2 proteins, AEYDRVHIYY (SEQ ID NO: 1), RLYPDAPPLR (SEQ ID NO: 33), RLGPADRRFVALSGS (SEQ ID NO: 12), and RPRGEVRFL (SEQ ID NO: 34) (FIG. 12) in a T cell activation assay using bulk biopsy T cells as responder cells and the indicated HLA-expressing cells as antigen presenting cells.

HSV-2 proteome-wide CD4 T_{RM} screens were performed as described by Johnston et al. (2014) and in the description of FIG. 13. FIG. 13 shows reactive HSV-2 proteins (UL1, UL19, UL23, UL29, and UL 36) that highly activate CD4 T cells.

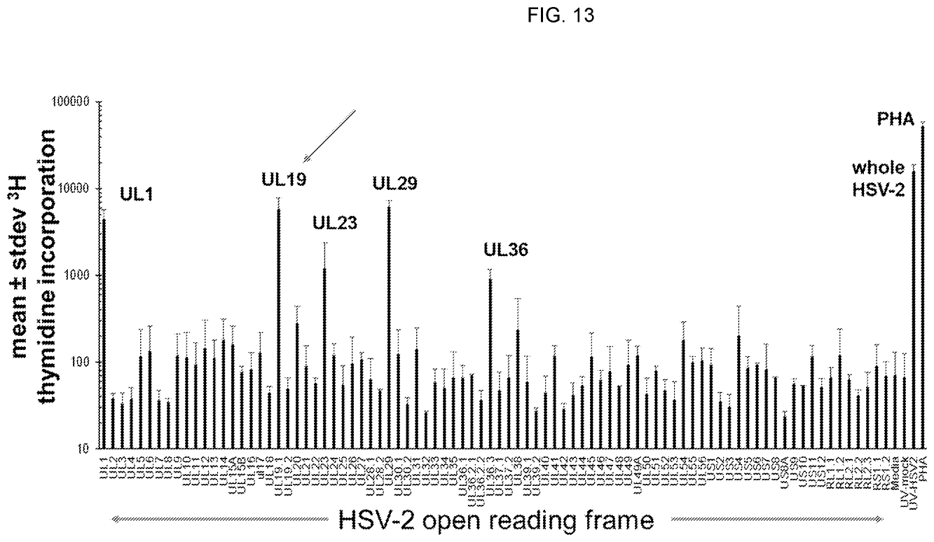

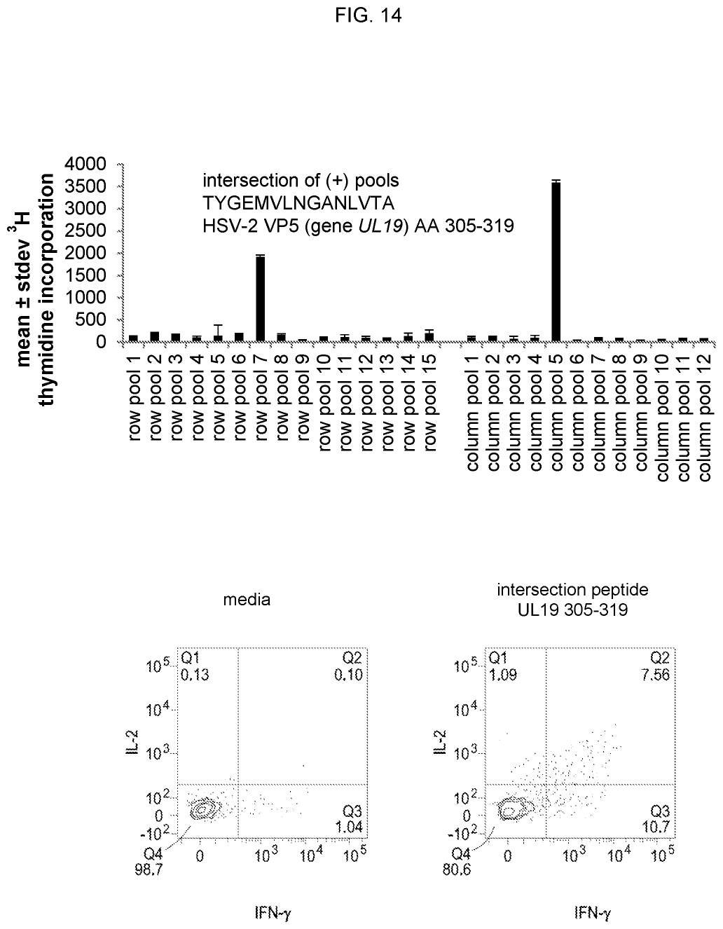

Biopsy-derived CD4 T cells recognizing Cos7 co-transfected with the combination of a subject HLA molecule and HSV-2 UL19 gene were identified. A matrix of UL19 peptide pools was created and tested for their ability to activate the biopsy-derived cells. One row and one column pool were positive (FIG. 14). The single peptide at the intersection of the row and column pools (‘UL19 305-319’, TYGEMVLNGANLVTA (SEQ ID NO: 14)) elicited an IFN-γ response from the biopsy cells (bottom, right-hand graph of FIG. 14) while the negative media control did not (bottom, left-hand graph of FIG. 14).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in ability to elicit or increase CD8 TRM at a site of recurrent HSV-2 infection.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. A composition comprising: (i) a first HSV-2 vaccine epitope of SEQ ID NO: 5 and/or SEQ ID NO: 6; and (ii) a second HSV-2 vaccine epitope selected from SEQ ID NOs: 13-22.

2. A composition of claim 1, wherein the first HSV-2 vaccine epitope is SEQ ID NO: 5.

3. A composition of claim 1, wherein the first HSV-2 vaccine epitope is SEQ ID NO: 6.

4. A composition of claim 1, wherein the first HSV-2 vaccine epitope comprises SEQ ID NOs: 5 and 6.

5. A composition of claim 1, wherein the first HSV-2 vaccine epitope and the second HSV-2 vaccine epitope are in one or more fusion proteins.

6. A composition of any one of claims 1-5, wherein the composition is an immunogenic composition.

7. A composition of any one of claims 1-5, wherein the composition is a therapeutic composition.

8. A composition of any one of claims 1-7, further comprising one or more adjuvants.

9. A composition of claim 8, wherein the one or more adjuvants are selected from alum, a squalene-based adjuvant, a STING agonist, a liposome-based adjuvant, a saponin-based adjuvant, a stable emulsion of TLR4 agonist glucopyranosyl lipid A (GLA SE), or a carbomer-lecithin-based adjuvant.

10. An HSV-2 vaccine epitope including one or more immunogenic proteins selected from SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12;
SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 30;
SEQ ID NO: 31; SEQ ID NO: 32; SEQ ID NO: 33; or SEQ ID NO: 34.

11. A fusion protein comprising an HSV-2 vaccine epitope of claim 10.

12. A fusion protein of claim 11 comprising at least 2 HSV-2 vaccine epitopes of claim 10; at least 3 HSV-2 vaccine epitopes of claim 10; at least 4 HSV-2 vaccine epitopes of claim 10 or at least at least 5 HSV-2 vaccine epitopes of claim 10.

13. A fusion protein of claim 11 comprising a CD8 TRM epitope and a CD4 TRM epitope.

14. A fusion protein of claim 13 comprising at least two CD8 TRM epitopes and a CD4 TRM epitope.

15. A fusion protein of claim 13 comprising a CD8 TRM epitope and at least two CD4 TRM epitopes.

16. A fusion protein of claim 11 comprising a multimerization domain.

17. A fusion protein of claim 16 wherein the multimerization domain is a C4b domain.

18. A fusion protein of claim 16 wherein the multimerization domain is selected from SEQ ID NO:
23; SEQ ID NO: 24; SEQ ID NO: 25; or SEQ ID NO: 26.

19. A composition comprising an HSV-2 vaccine epitope of claim 10.

20. A composition of claim 19 wherein the composition is an immunogenic composition.

21. A composition of claim 19 wherein the composition is a therapeutic composition.

22. A composition of claim 19 further including one or more adjuvants.

23. A composition of claim 22 wherein the one or more adjuvants are selected from alum, a squalene-based adjuvant, a STING agonist, a liposome-based adjuvant, a saponin-based adjuvant, a stable emulsion of TLR4 agonist glucopyranosyl lipid A (GLA SE), or a carbomer-lecithin-based adjuvant.

24. A method of stimulating an anti-HSV-2 immune response in a subject comprising administering to the subject a therapeutically effective amount of a composition of claim 19 thereby stimulating an HSV-2 immune response in the subject.

25. A method of claim 24 wherein the subject is HSV-2 seropositive.

26. A method of claim 24 wherein the subject is an HSV-2 seronegative subject, and wherein the therapeutically effective amount reduces the likelihood of the HSV-2 seronegative subject becoming HSV-2 seropositive.

27. A method of treating a herpes simplex virus type 2 (HSV-2) infection in a subject comprising administering the composition of claim 21 to the subject thereby treating the HSV-2 infection in the subject.

28. A method of eliciting CD8 and/or CD4 TRM comprising administering an HSV-2 vaccine epitope of claim 10.

29. A method of enhancing proliferation of HSV-specific T cells comprising contacting the HSV-specific T cells with an HSV-2 vaccine epitope of claim 10.

30. A method of inducing an immune response to an HSV infection in a subject comprising administering a composition of claim 19 to the subject thereby inducing an immune response to the HSV infection in the subject.
